SEQUENCE LISTING (5 sequences)

SEQ ID NO: 1 - protein, 127 aa, Camelus dromedarius
  REGION 1..127: note = immunoglobulin heavy chain variable region, partial
DVQLVESGGGSVQGAAGGSLRLSCAASDITYSTDCMGWFRQAPGKEREGVATINNGRAITYYADSVKGRFTISQDNAKNTVYLQMNSLRPKDTAIYYCAARLRAGYCYPADYSMDYWGKGTQVTVSS

SEQ ID NO: 2 - protein, 127 aa, Camelus dromedarius
  REGION 1..127: note = immunoglobulin heavy chain variable region, partial; note = immunoglobulin heavy chain variable region, partial
DVQLEESGGGSVQAGGSLRLSCAASGYIYSTYCMGWFRQAPGKEREGVAAINDVGGSVYYADSVKGRFTISQDIAQDTMYLQMNDLTPENTVTYTCAALRCLSDSDPDTRVHMYYDWGQGTQVTVSS

SEQ ID NO: 3 - protein, 125 aa, Camelus dromedarius
  REGION 1..125: note = immunoglobulin heavy chain variable region, partial
DVQLEESGGGSVQTGGSLRLSCAASGYTYSSACMGWFRQGPGKEREAVADVNTGGRRTYYADSVKGRFTISQDNTKDMRYLQMNNLKPEDTATYYCATGPRRRDYGLGPCDYNYWGQGTQVTVSS

SEQ ID NO: 4 - protein, 112 aa, Camelus dromedarius
  REGION 1..112: note = immunoglobulin heavy chain variable region, partial
EVQLEESGGGLVQPGGSLTLSCAASGYTFTNCAAGWYRQAPGKECELVASIFSGNRTNYADSVKGRFTISRDNTKDIVYLQMNSLKPEDTTVYYCDARTPCWGQGTQVTVSS

SEQ ID NO: 5 - protein, 121 aa, Camelus dromedarius
  REGION 1..121: note = immunoglobulin heavy chain variable region, partial
EVQLEESGGGSVQAGGSLRLSCAASGYTFLQLLHGWFRQAPGKEREVVARFNTDINKTFYLESVKGRFTLSQDNAKNTLYLQMNSLKPEDTAIYYCAASRPDSTCDYFAYRGQGTQVTVSS

(19) United States
(12) Patent Application Publication
(10) Pub. No.: US 2025/0073359 A1
(43) Pub. Date: Mar. 6, 2025

(54) RADIOLABELED BIOMOLECULES AND THEIR USE

(71) Applicant: DUKE UNIVERSITY, Durham, NC (US)

(72) Inventors: Michael Rod Zalutsky, Chapel Hill, NC (US); Ganesan Vaidyanathan, Chapel Hill, NC (US)

(21) Appl. No.: 18/946,772

(22) Filed: Nov. 13, 2024

Related U.S. Application Data
(62) Division of application No. 16/498,264, filed on Sep. 26, 2019.
(60) Provisional application No. 62/478,754, filed on Mar. 30, 2017.
(60) Provisional application No. 62/500,692, filed on May 3, 2017.
(60) Provisional application No. 62/529,532, filed on Jul. 7, 2017.
(60) Provisional application No. 62/583,134, filed on Nov. 8, 2017.

(51) Int. Cl. A61K 51/10; A61K 51/04; A61P 35/00

(52) U.S. Cl. CPC A61K 51/1051; A61K 51/0478; A61P 35/00

(57) ABSTRACT

The application is drawn to radiolabeled biomolecules and methods for radiolabeling biomolecules with radioactive halogen atoms that minimizes loss of the radioactive halogen due to dehalogenation in vivo, preserves the biological activity of the biomolecule, maximizes retention of radioactivity in cancer cells, and minimizes the retention of radioactivity in normal tissues after in vivo administration. Some such radiolabeled biomolecules comprise a radioactive metal atom in place of, or in addition to the radioactive halogen. The biomolecules have an affinity for particular types of cells and may specifically bind a certain cell, such as cancer cells. Relevant biomolecules include antibodies, monoclonal antibodies, antibody fragments, peptides, other proteins, nanoparticles and aptamers.

### CROSS-REFERENCE TO RELATED APPLICATIONS

The present application is a divisional of U.S. patent application Ser. No. 16/498,264, filed Sep. 26, 2019, which is a U.S. National Stage of International Patent Application PCT/IB2018/052211, filed Mar. 29, 2018, and claims priority to U.S. Provisional Patent Application Nos. 62/478,754, filed Mar. 30, 2017; 62/500,692, filed May 3, 2017; 62/529,532, filed Jul. 7, 2017; and 62/583,134, filed Nov. 8, 2017. The disclosures of each of the applications noted above are incorporated herein by reference in their entireties.

### SEQUENCE LISTING

The present application contains a Sequence Listing, which has been submitted electronically in ASCII format and is hereby incorporated by reference in its entirety. Said ASCII copy, created on Nov. 12, 2024, is named D571751030USPCTD1 Seq List.xml, and is 8 kilobytes in size.

### FIELD OF THE INVENTION

The present invention is drawn to compounds useful for radiolabeling biomolecules and to precursors thereof, as well as to radiolabeled biomolecules. The compounds can effectively retain radioactivity from biomolecules that become internalized within cells, rendering such compounds useful in the diagnosis and treatment of disease, particularly cancer.

### BACKGROUND

Radioiodination is one of the simplest ways to radiolabel a biomolecule. Several radioisotopes of iodine are available for imaging and targeted radiotherapy of cancer. Radioisotopes of iodine are supplied as alkaline solutions and iodine is present in these in an oxidation state of −1 (I; iodide). The standard method for biomolecule radioiodination requires oxidation of the iodine to the +1 oxidation state for electrophilic substitution into tyrosine amino acids present in biomolecules such as antibodies, other proteins and peptides. Challenges of thus radioiodinated monoclonal antibodies (mAbs) and peptides include their instability in vivo to proteolysis inside cells after internalization, deiodination, and as a consequence of both processes, loss of radioactivity from tumor cells. It is widely recognized that radioiodinated antibodies and peptides are proteolytically degraded inside cells after internalization (which can occur as a consequence of binding to receptors and certain antigens), to radioiodotyrosine that is efficiently exported from the cells by membrane amino acid transporters. Released radioiodotyrosine is deiodinated by deiodinases found in tissues and the free radioiodine redistributes and accumulates in organs with sodium iodide symporter expression, particularly the thyroid, stomach, and salivary glands. Thus, the amount of radiolabel that is retained in tumors is diminished and concomitantly, the uptake of radioactivity in normal tissues is increased.

One of the disadvantages of antibodies is their long half-life in the bloodstream, which results in high background levels after systemic administration and, consequently, in low tumor to background ratios. Moreover, conventional antibodies have a rather slow diffusion into solid tumors, which prevents them from reaching and binding to receptor/antigen in the entire tumor mass homogeneously.

While some compounds have been identified in the art, they are unstable and hard to produce in commercial quantities. Therefore, there is a need for improved prosthetic compounds that can be used to radiolabel biomolecules for targeted radiotherapies and imaging applications.

Moreover, the uptake of antibodies into tumor cells, particularly brain metastases, is low due to the size of the antibodies which is particularly problematic for tumors in the brain because of delivery restrictions imposed by the blood brain barrier. The present invention addresses the problems associated with the treatment of cancer, including cancer that has metastasized to the brain by compositions that are capable of being taken up and retained by the tumor cells, while reducing the amount of the radiolabel that is taken up by normal tissue, particularly the kidneys.

### SUMMARY OF THE INVENTION

The invention is drawn to methods, compounds, and compositions for radiolabeling biomolecules (also referred to as macromolecules) with radioactive halogen atoms in a manner which minimizes loss of the radioactive halogen due to dehalogenation in vivo, preserves the biological activity of the biomolecule, maximizes retention in diseased cells, such as cancer cells, and minimizes the retention of radioactivity in normal tissues after in vivo administration. The biomolecules have an affinity for particular types of cells. That is, the biomolecules may specifically bind a certain cell, such as cancer cells. Compositions of the invention include the radiolabeled biomolecules. Such biomolecules include antibodies, monoclonal antibodies, antibody fragments, peptides, other proteins, nanoparticles and aptamers. Such examples of biomolecules for purposes of the invention include, diabodies, scFv fragments, DARPins, fibronectin type III-based scaffolds, affibodies, VHH molecules (also, known as single domain antibody fragments (sdAb) and nanobodies), nucleic acid or protein aptamers, and nanoparticles. Additionally, larger molecules such as proteins >50 kDa including antibodies, monoclonal antibodies, chimeric antibodies, humanized antibodies, and F(ab′)₂ fragments can be used in the practice of the invention. In addition, nanoparticles with a size less than 50 nm can be used in the practice of the invention.

The methods of the invention utilize prosthetic compounds that are effective for radiolabeling. As such, the disclosure provides such radiolabeling compounds (referred to herein as “prosthetic compounds”), as well as precursors to afford such prosthetic compounds (referred to herein as “radiohalogen precursors”). The disclosure further provides radiolabeled macromolecules (e.g., biomolecules) comprising such prosthetic compounds/radicals and one or more macromolecules. In some such embodiments, these radiolabeled macromolecules are targeted radiotherapeutic agents. The prosthetic compounds and radiolabeled compounds of the invention are useful, e.g., for diagnosing disease and for targeted radiotherapy.

In one aspect of the present disclosure is provided a compound in the form of a prosthetic compound or radiohalogen precursor represented by Formula 1:

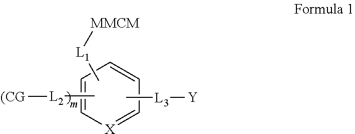

wherein:
X is CH or N;
L₁ and L₃ are independently selected from a bond, a substituted or unsubstituted alkyl chain, a substituted or unsubstituted alkenyl chain, a substituted or unsubstituted alkynyl chain, and a polyethylene glycol (PEG) chain;
MMCM is a macromolecule conjugating moiety;
L₂ is a substituted or unsubstituted alkyl chain, a substituted or unsubstituted alkenyl chain, a substituted or unsubstituted alkynyl chain, or a polyethylene glycol (PEG) chain comprising at least three oxygen atoms, wherein L₂ optionally contains a Brush Border enzyme-cleavable peptide;
CG is selected from guanidine; PO₃H; SO₃H; one or more charged D- or L-amino acids, such as arginine, phosphono/sulfo phenylalanine, glutamate, aspartate, and lysine; a hydrophilic carbohydrate moiety; a polyethylene glycol (PEG) chain; and Z-guanidine (also referred to herein as “guanidino-Z”);
Z is (CH₂)ₙ;
n is greater than 1;
m is 0 to 4 (where X═CH) or 0 to 3 (where X═N); and
Y is an alkyl metal moiety (in the radiohalogen precursor) or a radioactive halogen (in the prosthetic compound), wherein the radioactive halogen is selected from the group consisting of ⁷⁵Br, ⁷⁶Br, ⁷⁷Br, ¹²³I, ¹²⁴I, ¹²⁵I, ¹³¹I ²¹¹At, or a pharmaceutically acceptable salt or solvate thereof.

In certain preferred embodiments, m=1.

In some embodiments, Y is an alkyl metal moiety (where the compound is a radiohalogen precursor), selected from the group consisting of trimethyl stannyl (SnMe₃), tri-n-butylstannyl (SnBu₃) and trimethylsilyl (SiMe₃). In other embodiments, Y is a radioactive halogen (where the compound is a prosthetic compound) selected from the group consisting of ⁷⁵Br, ⁷⁶Br, ⁷⁷Br, ¹²³I, ¹²⁴I, ¹²⁵I and ²¹¹At.

In some embodiments, MMCM is an active ester or (Gly)ₘ, wherein m is 1 or more. In some embodiments, MMCM is selected from the group consisting of N-hydroxysuccinimide (NHS) ester, tetrafluorophenol (TFP) ester, an isothiocyanate group, or a maleimide group. One exemplary MMCM is Gly-Gly-Gly.

In some embodiments, L₂ is (CH₂)ₚ, wherein p=1 to 6 or wherein p=2 to 6. The optional Brush Border enzyme-cleavable peptide, where present within L₂, is selected in some embodiments from the group consisting of Gly-Lys, Gly-Tyr and Gly-Phe-Lys.

In certain embodiments, the compound (prosthetic compound or radiohalogen precursor) is represented by the following structure of Formula 1a:

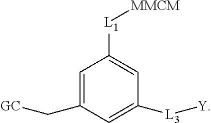

In certain embodiments, the compound comprises N-succinimidyl 3-guanidinomethyl-5-[¹³¹I]iodobenzoate (iso-[¹³¹I]SGMIB), or N-succinimidyl 3-[²¹¹At]astato-5-guanidinomethyl benzoate (iso-[²¹¹At]SAGMB).

In another aspect of the invention, the disclosure provides a compound in the form of a prosthetic compound or radiohalogen precursor represented by Formula 2:

MC—Cm—L₄—Cm—T Formula 2,
wherein:
MC is a polydentate metal chelating moiety;
Cm is thiourea, amide, or thioether;
L₄ is selected from a bond, a substituted or unsubstituted alkyl chain, a substituted or unsubstituted alkenyl chain, a substituted or unsubstituted alkynyl chain, optionally having NH, CO, or S on one or both termini, and a polyethylene glycol (PEG) chain; and
T is a compound (prosthetic compound or radiohalogen precursor) as disclosed herein (e.g., according to Formula 1, e.g., Formula 1A),
or a pharmaceutically acceptable salt or solvate thereof.

In some embodiments, MC is a macrocyclic structure. In certain exemplary prosthetic compounds, MC is selected from DOTA, TETA, NOTP, and NOTA. In some embodiments, MC is an acyclic polydentate ligand. In certain exemplary prosthetic compounds, MC is selected from EDTA, EDTMP, and DTPA.

In certain embodiments, Y is an alkyl metal moiety (where the compound is a radiohalogen precursor). The alkyl metal moiety in the radiohalogen precursor is, for example, selected from the group consisting of trimethyl stannyl (SnMe₃), tri-n-butylstannyl (SnBu₃) and trimethylsilyl (SiMe₃). Such precursors, as will be described herein, can be useful in producing the prosthetic compounds and radiolabeled biomolecules disclosed herein. In other embodiments, Y is a radioactive halogen (where the compound is a prosthetic compound), such as ⁷⁵Br, ⁷⁶Br, ⁷⁷Br, ¹²³I, ¹²⁴I, ¹²⁵I, ¹³¹I or ²¹¹At.

The disclosure further provides a radiolabeled biomolecule, comprising a prosthetic compound as disclosed herein attached to a biomolecule and also provides an intermediate, comprising a radiohalogen precursor as disclosed herein attached to a biomolecule, which can be reacted to form a radiolabeled biomolecule.

The biomolecule can vary. In certain embodiments, the biomolecule is selected from the group consisting of an antibody, an antibody fragment, a VHH molecule, an aptamer or variations thereof. In certain embodiments, the biomolecule is a VHH. The VHH, in particular embodiments, targets HER2. In some embodiments, the VHH comprises an amino acid sequence selected from the sequences set forth in SEQ ID NOs: 1-5.

The disclosure further provides a pharmaceutical composition comprising a radiolabeled biomolecule as disclosed herein in association with a pharmaceutically acceptable adjuvant, diluent or carrier. In a further aspect of the disclosure is provided a method of treatment for cancer, comprising administering to an individual in need thereof an effective amount of a radiolabeled biomolecule as disclosed herein and/or an effective amount of a pharmaceutical composition as disclosed herein.

### BRIEF DESCRIPTION OF THE DRAWINGS

In order to provide an understanding of embodiments of the current disclosure, reference is made to the appended drawings, which are not necessarily drawn to scale. The drawings are exemplary only, and should not be construed as limiting the disclosure.

FIG. 1 provides non-reducing SDS-PAGE/phosphor imaging profiles of (A) [²¹¹At]SAGMB-5F7 VHH, (B) [¹³¹I]SGMIB-5F7 VHH, (C) iso-[²¹¹At]SAGMB-5F7 VHH, and (D) iso-[¹³¹I]SGMIB-5F7 VHH, with molecular weight standards in left lane for comparison;

FIG. 2 provides the results of saturation binding assays performed on HER2-expressing BT474M1 breast carcinoma cells with 5F7 VHH labeled using (A) [¹³¹I]SGMIB, (B) iso-[¹³¹I]SGMIB, (C) [²¹¹At]SAGMB and (D) iso-[²¹¹At]SAGMB;

FIG. 3 provides plots of internalization of [²¹¹At]SAGMIB-5F7 VHH and iso-[²¹¹At]SAGMB-5F7 VHH in BT474M1 cells in vitro, with FIG. 3A depicting total cell-associated (internalized+surface-bound) radioactivity and FIG. 3B depicting internalized radioactivity;

FIG. 4 provide plots of internalization of [¹³¹I]SGMIB-5F7 VHH and iso-[¹³¹I]SGMIB-5F7 VHH in BT474M1 cells in vitro, with FIG. 4A showing total cell-associated (internalized+surface-bound) radioactivity and FIG. 4B showing internalized radioactivity;

FIG. 5 depicts biodistribution of [²¹¹At]SAGMB-5F7 VHH and iso-[²¹¹At]SAGMB-5F7 VHH in SCID mice bearing BT474M1 xenografts, with a comparison of uptake in tumor, with data obtained from paired-label studies after administering [¹³¹I]SGMIB-5F7/[²¹¹At]SAGMB-5F7 VHH and iso-[¹³¹I]SGMIB-57/iso-[²¹¹At] SAGMB-5F7 VHH tandems;

FIG. 6 depicts biodistribution of [¹³¹I]SGMIB-5F7 VHH and iso-[¹³¹I]SGMIB-5F7 VHH in SCID mice bearing BT474M1 xenografts: comparison of uptake in tumor, with data obtained from paired-label studies after administering [¹³¹I]SGMIB-5F7/[²¹¹At]SAGMB-5F7 VHH and iso-[¹³¹I]SGMIB-57/iso-[²¹¹At]SAGMB-5F7 VHH tandems;

FIG. 7 depicts biodistribution of [²¹¹At]SAGMB-5F7 and iso-[²¹¹At]SAGMB-5F7 VHH in SCID mice bearing BT474M1 xenografts: comparison of uptake in kidneys, with data obtained from paired-label studies after administering [¹³¹I]SGMIB-5F7/[²¹¹At]SAGMB-5F7 VHH and iso-[¹³¹I]SGMIB-57/iso-[²¹¹At]SAGMB-5F7 VHH tandems;

FIG. 8 depicts biodistribution of [¹³¹I]SGMIB-5F7 VHH and iso-[¹³¹I]SGMIB-5F7 VHH in SCID mice bearing BT474M1 xenografts: comparison of uptake in kidneys, with data obtained from paired-label studies after administering [¹³¹I]SGMIB-5F7/[²¹¹At]SAGMB-5F7 VHH and iso-[¹³¹I]SGMIB-57/iso-[²¹¹At]SAGMB-5F7 VHH tandems;

FIG. 9 provides data on uptake of [²¹¹At]SAGMB-5F7 VHH and iso-[²¹¹At]SAGMB-5F7 VHH in thyroid (FIG. 9A) and stomach (FIG. 9B) in SCID mice bearing BT474M1 xenografts, with data obtained from paired-label studies after administering [¹³¹I]SGMIB-5F7/[²¹¹At]SAGMB-5F7 VHH and iso-[¹³¹I]SGMIB-57/iso-[²¹¹At]SAGMB-5F7 VHH tandems;

FIG. 10 provides data on uptake of [¹³¹I]SGMIB-5F7 and iso-[¹³¹I]SGMIB-5F7 in thyroid (FIG. 10A) and stomach (FIG. 10B) in SCID mice bearing BT474M1 xenografts, with data obtained from paired-label studies after administering [¹³¹I]SGMIB-5F7/[²¹¹At]SAGMB-5F7 VHH and iso-[¹³¹I]SGMIB-57/iso-[²¹¹At]SAGMB-5F7 VHH tandems;

FIG. 11 depicts tumor-to-tissue ratios obtained from the biodistribution of [²¹¹At]SAGMB-5F7 VHH and iso-[²¹¹At]SAGMB-5F7 VHH in SCID mice bearing BT474M1 xenografts; with data obtained from paired-label studies after administering [¹³¹I]SGMIB-5F7/[²¹¹At]SAGMB-5F7 VHH and iso-[¹³¹I]SGMIB-5F7/iso-[²¹¹At]SAGMB-5F7 VHH tandems; and

FIG. 12 depicts tumor-to-tissue ratios obtained from the biodistribution of [¹³¹I]SGMIB-5F7 VHH and iso-[¹³¹I]SGMIB-5F7 in SCID mice bearing BT474M1 xenografts, with data obtained from paired-label studies after administering [¹³¹I]SGMIB-5F7/[²¹¹At]SAGMB-5F7 VHH and iso-[¹³¹I]SGMIB-5F7/iso-[²¹¹At]SAGMB-5F7 VHH tandems;

FIG. 13 is a table providing paired label biodistribution of [²¹¹At]SAGMB-5F7 VHH and [¹³¹I]SGMIB-5F7 VHH in SCID mice with subcutaneous B474M1 human breast carcinoma xenografts; and

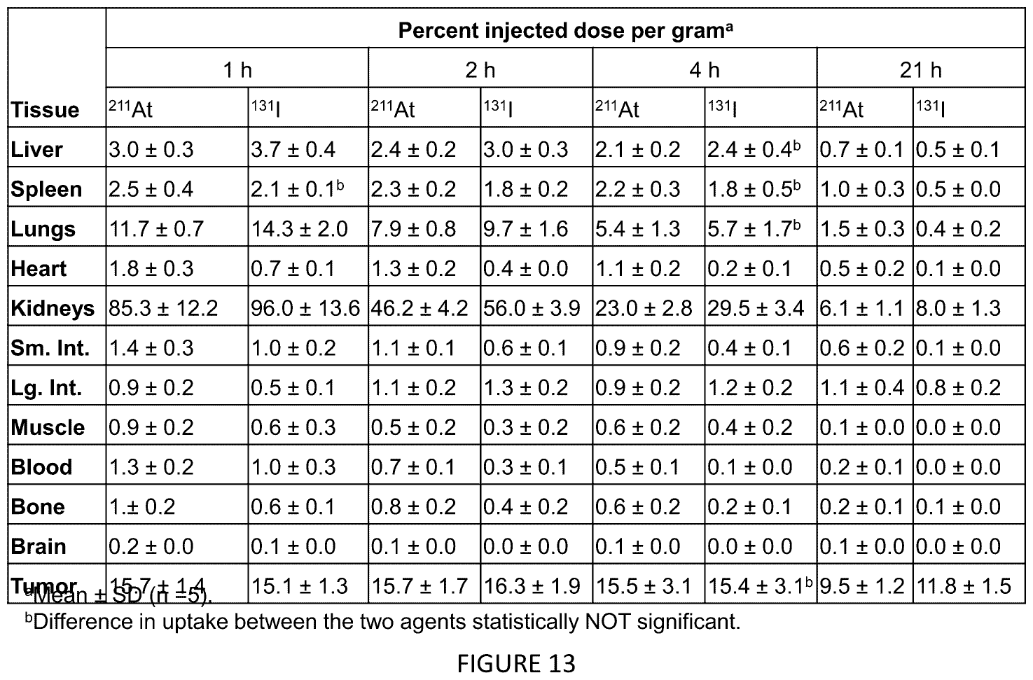

FIG. 14 is a table providing paired label biodistribution of iso-[²¹¹At]SAGMB-5F7 VHH and iso-[¹³¹I]SGMIB-5F7 VHH in SCID mice with subcutaneous B474M1 human breast carcinoma xenografts.

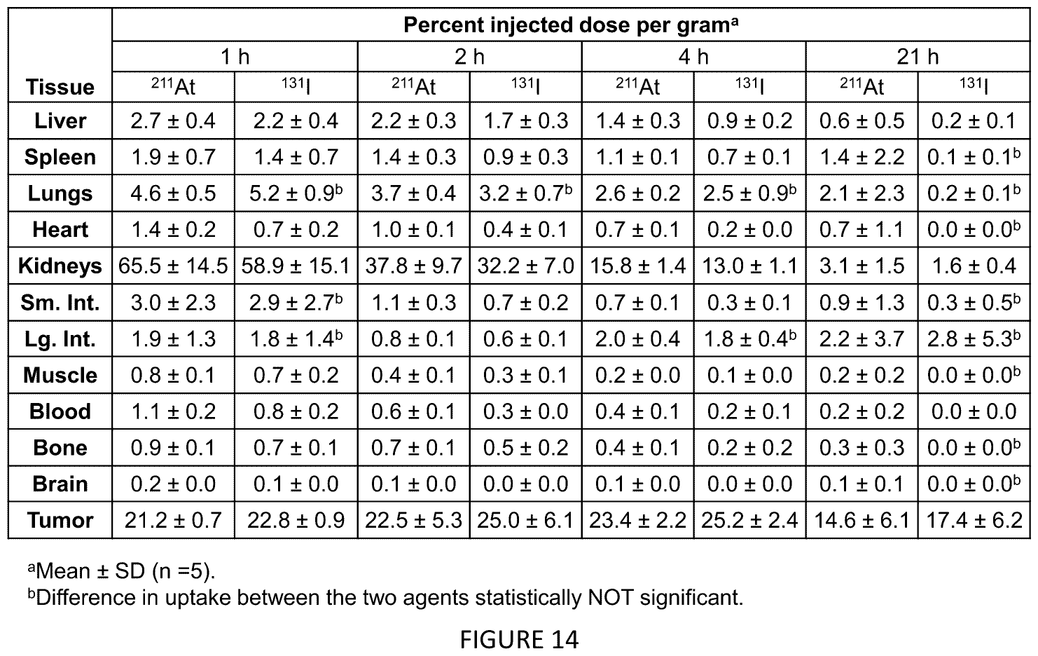

### DETAILED DESCRIPTION OF THE INVENTION

The present disclosure will now be described more fully hereinafter with reference to exemplary embodiments thereof. These exemplary embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

Compounds, compositions and methods for diagnosing and treating disease including cancer are provided. Generally, compounds of the present disclosure comprise a radiolabeled prosthetic compound/radical or a radiolabeled prosthetic group attached to a macromolecule, e.g., a biomolecule that serves as a targeting moiety (providing a targeted radiotherapeutic agent). As such, the present disclosure encompasses radiolabeled prosthetic compounds and radicals themselves, as well as macromolecules having such radiolabeled prosthetic compounds/radicals attached thereto (which are referred to in some embodiments herein as “radiolabeled biomolecules” or “targeted radiotherapeutic agents”).

The disclosure also encompasses such compounds and radicals (alone and/or in combination with a biomolecule) containing an alkyl metal moiety (referred to herein as “radiohalogen precursors”) from which a prosthetic group and/or a targeted radiotherapeutic agent can be produced. Advantageously, in some embodiments, preparation of such precursors allows for the production of prosthetic compounds, as well as targeted radiotherapeutic agents, comprising larger radioactive halogens (e.g., larger than ¹⁸F, including, but not limited to, ⁷⁵Br, ⁷⁶Br, ⁷⁷Br, ¹²³I, ¹²⁴I, ¹²⁵I, ¹³¹I and ²¹¹At).

A labeled prosthetic compound/radical or a radiohalogen precursor (alone or attached to a macromolecule) generally includes, in addition to a radioactive halogen or precursor thereto, a charged group (CG), and a macromolecule conjugating moiety (MMCM). Each of these components can be associated with one or more cleavable (or non-cleavable) linkers, as will be described in more detail below. The targeted radiotherapeutic agent, in some embodiments, comprises a biomolecule (targeting moiety), a radiolabeled prosthetic group or template, and, optionally, a chelating agent (either macrocyclic or acyclic).

The radiolabeled compounds and, in particular, the radiolabeled biomolecules and the methods of use described herein, result in greater uptake of the radioactivity in the targeted cells, higher retention of radioactivity in the targeted cells after internalization, and less uptake of the radioactivity in normal cells; for example, there is less thyroid and renal uptake of the radioactivity. The targeted radiotherapy of the invention is capable of selectively delivering a radionuclide to malignant cell populations. An advantage of targeted radiotherapy is that one can select a radionuclide with properties that are best matched to the constraints of the intended clinical application. As one example, for central nervous system (CNS) tumors, radiation would advantageously be selected with a tissue range that minimizes irradiation of normal CNS tissues.

The compounds provided herein (e.g., the radiohalogen precursors, prosthetic compounds, intermediates, and the targeted radiotherapeutic agents) are prepared by a method that enhances the retention of a radionuclide, particularly (in certain embodiments), a radiohalogen, in targeted diseased cells, such as cancer cells, using labeling techniques that generate a charged catabolite, following intracellular proteolysis, which cannot traverse the lysosomal or cell membrane and is resistant to exocytosis. The compounds of the invention comprise a charged catabolite where the portion of the molecule bearing the label is inert to lysosomal degradation and becomes trapped inside the cell after proteolysis.

Certain prosthetic compounds and precursors thereto (i.e., radiohalogen precursors) encompassed by the present disclosure include those of Formula 1 and derivatives and variants thereof.

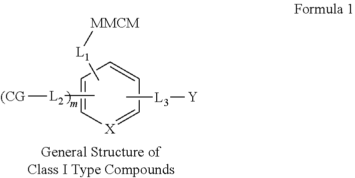

The invention includes prosthetic compounds/radicals and precursors thereof with the general structure of Formula 1 (referred to as “Class I Type Compounds”), which comprise a homo (X═CH) or hetero (X =N) aromatic ring having attached thereto: a macromolecule conjugating moiety (MMCM) to couple the prosthetic compound/radical or precursor to a macromolecule, a radioactive halogen or a radiohalogen precursor (Y); and one or more charged substituents/groups (CG). Each of these components can be attached to the aromatic ring through a linker (L₁, L₂, L₃) or can be directly bonded to the aromatic ring (i.e., where L₁ and/or L₂ and/or L₃ is a bond). Each of these components shown in Formula 1 will be described in further detail below.

In some embodiments, Y is a radioactive halogen (where Formula 1 represents a radiolabeled prosthetic compound/radical). Such radioactive halogens can be selected from ¹⁸F, ⁷⁵Br, ⁷⁶Br, ⁷⁷Br, ¹²³I, ¹²⁴I, ¹²⁵I, ¹³¹I, and ²¹¹At. Advantageously, the radioactive halogens in some embodiments are larger than ¹⁸F. In certain embodiments, the radioactive halogen Y is selected from ⁷⁵Br, ⁷⁶Br, ⁷⁷Br, ¹²³I, ¹²⁴I, ¹²⁵I, ¹³¹I, and ²¹¹At. In certain embodiments, the radioactive halogen Y is selected from ⁷⁵Br, ⁷⁶Br, ⁷⁷Br and ²¹¹At. In one particular embodiment, the radioactive halogen Y is ²¹¹At.

In other embodiments, Y is an alkyl metal moiety (where Formula 1 represents a radiohalogen precursor/radical). Exemplary alkyl metal moieties include, but are not limited to, trialkyl metal precursors including trimethyl stannyl (SnMe₃), tri-n-butylstannyl (SnBu₃), and trimethylsilyl (SiMe₃).

Y can be directly bound to the aromatic ring (L₃=a direct bond) or can be bound to the aromatic ring through a linker (L₃). L₃ can be, e.g., a spacer such as a substituted or unsubstituted alkyl chain, a substituted or unsubstituted alkenyl chain, a substituted or unsubstituted alkynyl chain, or a short polyethylene glycol (PEG) chain (1-10 ethylene glycol units).

The charged group (CG) is typically present in the prosthetic groups disclosed herein, i.e., m is 1 or greater. Typically, m is 1; however, more than one CG can be attached to the ring such that m=2, m=3, and (where X═CH), m can be 4. Where more than one CG is attached to the ring, each such CG (and corresponding L₂) can be the same or different. In certain embodiments, as referenced below (as shown in Formula 2), another moiety can be attached to the ring of Formula 1 and, where such additional moiety is charged, m can be 0 (i.e., the additional moiety may, in some embodiments, effectively serve as the “charged group”).

The charged group is typically a group that is charged under the physiological conditions of the internal cell environment. In some embodiments, the charged group (CG) comprises a guanidine, a PO₃H group, or an SO₃H group. In some embodiments, CG is a guanidino-alkyl group containing more than one carbon. In some embodiments, CG is a guanidino-hydrophilic group (such as an amino-or hydroxyl-containing group), and/or an alkyloxycarbonylguanidine group. In other embodiments, CG comprises one or more charged D-amino acids such as arginine, glutamate, aspartate, lysine, and/or phosphono/sulfo phenylalanine. In still further embodiments, CG comprises a hydrophilic carbohydrate moiety. The compounds, in some embodiments, may contain one, two or three CG moieties (and, optionally, corresponding linker groups L₂) to increase intracellular trapping in cancer cells.

CG can be directly bound to the aromatic ring (L₂=a direct bond) or can be bound to the aromatic ring through a linker (L₂). L₂ can be, e.g., a spacer such as a substituted or unsubstituted alkyl chain (e.g., a simple substituted or unsubstituted alkyl chain such as a methylene), a substituted or unsubstituted alkenyl chain, a substituted or unsubstituted alkynyl chain, a PEG chain of at least three oxygens, or any of the foregoing containing a Brush Border enzyme-cleavable peptide such as Gly-Lys, Gly-Tyr or Gly-Phe-Lys. It is noted that, in certain embodiments, where CG is a guanidine and L₂ is an unsubstituted alkyl chain, the unsubstituted alkyl chain comprises two or more carbon atoms.

In some embodiments of the invention, a metabolizable spacer or cleavable linker, L₂ (e.g., a Brush Border enzyme cleavable linker), is located between CG and the aromatic ring. With these formulations, increased uptake and retention of radioactivity in the kidneys can be avoided as the CG moiety is cleaved off in the kidneys, eliminating the charge and allowing the radioactive species (now neutral or less charged) to escape from the renal tubule cells in the kidney after which they are rapidly excreted into the urine. While Brush Border enzyme cleavable linkers have been used before with radioactivity, they have not been used in this way to create a “charge switch” where the labeling reagent is charged in the tumor so is retained but loses charge in the kidney, so it is cleared.

Such linkers include linker sequences targeting meprin B, a metalloprotease expressed in the kidney brush-border membrane (Jodal et al. (2015) PLoS One April 9;10(4):e0123443); C-terminal lysines linked to antibody fragments via the epsilon-amino group of lysine or a C-terminal (N (epsilon)-amino-1,6-hexane-bis-vinyl sulfone) lysine that show reduced kidney uptake by taking advantage of the lysine specific carboxypeptidase activity of the kidney brush border enzymes that cleave off the radiolabeled peptide linker prior to uptake by proximal tubule cells (Li et al. (2002) Bioconjug Chem 13(5): 985-995); L-tyrosine O-methyl, L-asparagine, L-glutamine, N-Boc-L-lysine (Akizawa et al. (2013) Bioconjugate Chem 24:291-299); glycyl-lysine (Arano et al. (1999) Cancer Research 59:128-134); all of which are herein incorporated by reference.

In some embodiments, MMCM is an active ester. An active ester is defined herein as an ester that can be conjugated with amine groups present on a macromolecule/biomolecule (e.g., a peptide or protein) under mild conditions, i.e., conditions that will not result in loss of biological function of the macromolecule/biomolecule. Exemplary such MMCM groups include, but are not limited to, N-hydroxysuccinimide (NHS) or tetrafluorophenol (TFP) ester, an isothiocyanate group, or a maleimide group. Such MMCMs generally result in random (non-site specific) labeling of amine groups on the protein or peptide. In other embodiments, MMCM provides for site-specific conjugation to be performed using the enzyme Sortase, which results in conjugation to only one site (either the N-terminus or the C-terminus of the protein). In this case, MMCM is, e.g., the tripeptide GlyGlyGly.

MMCM can be directly bound to the aromatic ring (L₁=a direct bond) or can be bound to the aromatic ring through a linker (L₁). L₁ can be, e.g., a spacer such as a substituted or unsubstituted alkyl chain, a substituted or unsubstituted alkenyl chain, a substituted or unsubstituted alkynyl chain, or a short polyethylene glycol (PEG) chain (1-10 ethylene glycol units).

The positions of these three moieties (—L₁-MMCM, —L₂-CG, and —L₃—Y) on the aromatic ring can vary. Where X is CH, these three moieties, can be placed at any of the positions of the aromatic ring. In some such embodiments, the —L₂—CG, and —L₃—Y moieties are located at the 3 and 4 positions, respectively (or the 4 and 3 positions, respectively) relative to the —L₁-MMCM moiety (at the 1 position). In some such embodiments, the —L₂—CG, and —L₃—Y moieties are located at the 3 and 5 positions with respect to the —L₁-MMCM moiety, such that the aromatic ring comprises the referenced moieties at the 1, 3, and 5 positions. Where X is N, these three moieties can be placed at any of the remaining five positions of the ring, e.g., including, but not limited to, at the 2, 4, and 6 positions of the ring.

Certain prosthetic compounds within the scope of Formula 1 for labeling the targeting molecules of the invention, and radiohalogen precursors include compounds of Formula 1A and derivatives and variants thereof, as shown below. As shown, in Formula 1A, X is CH (i.e., the aromatic ring is a benzene ring), L₂ is a methylene group, and the three moieties (—L₁-MMCM, —L₃—Y, and —CH₂—CG) are present at the 1, 3, and 5 positions of the aromatic ring.

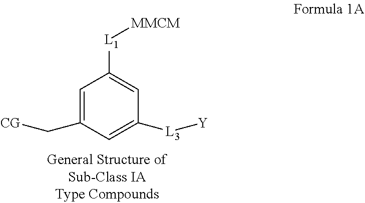

The invention also includes compounds thereof with the general structure of Formula 2 shown below (referred to as “Class II Type Compounds”).

MC—Cm—L₄—Cm—T

### Formula 2: General Structure of Class II Compounds

Such compounds include a polydentate metal chelating moiety (MC), a linker (L₄) with a conjugating moiety (Cm) at both ends of L₄, and a radiohalogenated template or radiohalogen precursor template (T). T can be, for example, a compound of Formula 1 or a compound of Formula 1A, as shown above (a compound containing a MMCM). In some embodiments, T is a prosthetic compound/radical and in some embodiments, T is a radiohalogen precursor compound/radical. In some such embodiments, as referenced above, m=0, where the “MC—Cm—L₄—Cm” moiety of Formula 2 provides the desired function of the L2-CG moiety in Formula 1, above (i.e., the MC-Cm-La-Cm substituent is a sufficiently “charged group”). In other such embodiments, m=1, 2, or 3, such that the aromatic ring of “T” has at least four substituents, i.e., L₁-MMCM, L₃—Y, L₂—CG, and Cm—L₄—Cm—MC, and may optionally comprise one or more additional L₂—CG substituents.

L₄ can be as defined above for L₁ and L₃. As such, L₄ can be a direct bond or can be, e.g., a spacer such as a substituted or unsubstituted alkyl chain, a substituted or unsubstituted alkenyl chain, a substituted or unsubstituted alkynyl chain, or a short polyethylene glycol (PEG) chain (1-10 ethylene glycol units). L₄ is again, as defined above but has NH, CO (carbonyl), or S (thioether) on one or both termini.

Cm can be, e.g., a thiourea, an amide, or a thioether. For example, in some embodiments, Cm is thiourea (e.g., when the conjugating functionality in the chelating moiety and T is an isothiocyanate), an amide (when the conjugating functionality in the chelating moiety and T is NHS or TFP active ester, or acyl halide), or thioether (when the conjugating functionality in the chelating moiety and T is maleimide).

T is generally a radiolabeled moiety or a radiohalogen precursor containing a MMCM via which a macromolecule can be coupled to the compound. As referenced above, T can, in some embodiments, be a compound/radical of Formula 1 or a compound/radical of Formula 1A. In other embodiments, other radiohalogen templates (T) can be used, including, but not limited to, iso-SGMIB, as disclosed in Choi et al. (2014) Nucl Med Biol 41(10): 802-812, which is incorporated herein by reference; SIPC, as disclosed in Reist et al. (1997) Nucl Med Biol 24(7): 639-648, which is incorporated herein by reference; or SDMB, as disclosed in U.S. Pat. No. 5,302,700, which is incorporated herein by reference.

MC can be any polydentate moiety and can be cyclic or acyclic. The composition of MC can vary. MC can be either uncomplexed (lacking a metal) or complexed with the stable (nonradioactive) or radioactive form of a metal, preferably a trivalent metal (M⁺³) such as lutetium, yttrium, indium, actinium, or gallium and the MC is connected to the linker either using one of the free COOH groups present on the MC or via other positions on the MC including one of the MC backbone carbons. Certain specific radioactive metals that can be complexed with the MC include, but are not limited to, radioactive metals selected from the group consisting of ¹⁷⁷Lu, ⁶⁴Cu, ¹¹¹In, ⁹⁰Y, ²²⁵Ac, ²¹³Bi, ²¹²Pb, ²¹²Bi, ⁶⁷Ga, ⁶⁸Ga, ⁸⁹Zr, and ²²⁷Th. It is noted that this list is not exhaustive and, although these exemplified radioactive metals are trivalent, certain MCs that may be used according to the present invention may bind metals of other valencies, and such MCs and radioactive metals are also encompassed herein.

In some embodiments, the inclusion of a radioactive metal associated with the MC can eliminate the need for a radioactive atom elsewhere on the molecule (e.g., as “Y” when T of Formula 2=a moiety of Formula 1/1a). As such, in Formula 2 compounds, “T” may or may not include a radioactive atom (e.g., halogen). In some embodiments, T comprises a moiety as shown in Formula 1/1a above, wherein the “Y” group is a non-radioactive halogen (e.g., a non-radioactive bromine or iodine). In other embodiments, a compound of Formula 2 is provided which comprises both a radiohalogen (e.g., as “Y” when T of Formula 2=a moiety of Formula 1/1a) and a radiometal (associated with MC, such as the radioactive metals referenced above). In certain particular embodiments, such a strategy would allow, e.g., for use of the same prosthetic agent for multiple isotopes. In certain specific examples, a compound of Formula 2 is provided with a low energy beta emitter (e.g., ¹³¹I) plus a high energy beta emitter (e.g., ⁹⁰Y); or an alpha emitter (e.g., ²²⁵Ac) metal and a beta emitter halogen (e.g., ¹³¹I); or an alpha emitter halogen (e.g., ²¹¹At) and a beta emitter radiometal (e.g., ¹⁷⁷Lu).

In some embodiments, MC is a macrocyclic ligand, consisting of a ring containing 8 or more atoms, bearing at least 3 negatively charged substituents such as carboxyl or phosphonate groups. Exemplary macrocyclic ligands suitable as the MC group include 1,4,7,10-tetraazacyclododecane-1,4,7,10-tetraacetic acid (DOTA), 1,4,7-triazacyclononane-1,4,7-triacetic acid (NOTA), 1,4,8,11-tetraazacyclotetradecane-1,4,8,11-tetraacetic acid (TETA), and 1,4,7-triazacyclononane-1,4,7-tri(methylene phosphonic acid) (NOTP). In other embodiments, MC is MeO-DOTA, as disclosed in Gali et al., Anticancer Research (2001), 21(4A), 2785-2792), which is incorporated herein by reference.

An example of a Class II compound is illustrated below in Formula 2A, wherein MC is a macrocyclic ligand comprising DOTA, and wherein the radiohalogenated template T is a moiety corresponding to Formula 1.

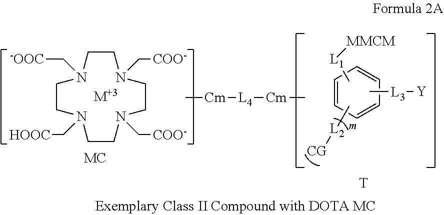

The left-hand brackets in Formula 2A are intended to convey that the specific site on the MC (DOTA) to which the Cm group is bonded is not limited, i.e., the Cm may be bonded to DOTA at various sites thereon. Similarly, the right-hand brackets in Formula 2A are intended to convey that the specific site on the ring of “T” to which the Cm group is bonded is not limited, i.e., Cm may be bonded to T at various sites on the ring. Again, as referenced above, CG—L₂ may or may not be present. In some embodiments, the benzene ring of T in Formula 2A comprises four substituents (including the linked MC, L₂-MMCM, L₃—Y, and L₂—CG). In other embodiments, the benzene ring of T in Formula 2A comprises three substituents (including the linked MC, L₂-MMCM, and L₃—Y). The latter embodiments are particularly relevant when the linked MC is charged, i.e., it can take the place in providing the desired function of the “L₂—CG” substituent.

In some embodiments, MC is an acyclic ligand, consisting of a chain containing 6 or more atoms bearing at least 3 negatively charged substituents such as carboxyl or phosphonate groups. Exemplary acyclic ligands suitable as the MC group include diethylenetriaminepentaacetic acid (DTPA), ethylenediaminetetramethylenephosphonic acid (EDTMP), and ethylenediaminetetraacetic acid (EDTA). An example of a Class II compound is illustrated below in Formula 2B, wherein MC is an acyclic ligand comprising DTPA, and wherein the radiohalogenated template T is a moiety corresponding to Formula 1.

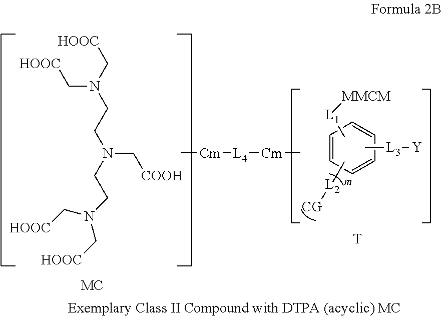

As referenced above with respect to Formula 2A, the left-hand brackets in Formula 2B are intended to convey that the specific site on the MC (DTPA) to which the Cm group is bonded is not limited, i.e., the Cm may be bonded to DTPA at various sites thereon. Similarly, the right-hand brackets in Formula 2B are intended to convey that the specific site on the ring of “T” to which the Cm group is bonded is not limited, i.e., Cm may be bonded to T at various sites on the ring. Again, as referenced above, CG—L₂ may or may not be present. In some embodiments, the benzene ring of T in Formula 2B comprises four substituents (including the linked MC, L₂-MMCM, L₃—Y, and L₂—CG). In other embodiments, the benzene ring of T in Formula 2A comprises three substituents (including the linked MC, L₂-MMCM, and L₃—Y). The latter embodiments are particularly relevant when the linked MC is charged, i.e., it can take the place in providing the desired function of the “L₂—CG” substituent.

In some specific embodiments, a compound of Formula 2 is provided, wherein MC=DOTA, L₄=—NH(CH₂)₆NH—, T=3-iodo-5-succinimidyloxycarbonyl-benzoyl, Cm=amide and MMCM=N-hydroxysuccinimide ester, a maleimide-containing moiety, or (Gly), for site-specific conjugation using Sortase (refer to the Formulas above).

It is noted that the formulas above, comprising a MMCM, can be further functionalized with an attached macromolecule (e.g., biomolecule) and as such, in some embodiments, compounds of any of the formulas provided herein above are encompassed, which further comprise a macromolecule (e.g., biomolecule) coordinated thereto via the MMCM. The disclosure thus encompasses intermediates (comprising a radioligand precursor and a biomolecule) and radiolabeled biomolecules (comprising a prosthetic group and a biomolecule), both of which may or may not comprise a metal chelating moiety.

The present disclosure further provides methods of synthesizing the prosthetic compounds and radiolabeled biomolecules described herein. In some embodiments, the methods generally comprise preparing a compound according to Formula 1 wherein Y=an alkyl metal radiohalogen precursor. In certain embodiments, the methods generally comprise preparing a compound according to Formula 2, wherein Y=an alkyl metal radiohalogen precursor. Employing such precursors, in some embodiments, allows for the preparation of prosthetic compounds and radiolabeled biomolecules comprising larger radioactive “Y” groups, e.g., larger than ¹⁸F, including, but not limited to, ⁷⁵Br, ⁷⁶Br, ⁷⁷Br, ¹²³I, ¹²⁴I, ¹²⁵I, ¹³¹I and ²¹¹At. In some embodiments, the macromolecule can be coordinated to the MMCM while Y is in the form of an alkyl metal radiohalogen precursor; then a subsequent reaction provides the product, wherein Y is in the form of the desired radioactive halogen atom.

### Definitions

“Cₘ-Cₙalkyl” on its own or in composite expressions such as Cₘ-Cₙhaloalkyl, Cₘ-Cₙalkylcarbonyl, Cₘ-Cₙalkylamine, etc. represents a straight or branched aliphatic hydrocarbon radical having the number of carbon atoms designated, e.g. C₁-C₄alkyl means an alkyl radical having from 1 to 4 carbon atoms. C₁-C₆alkyl has a corresponding meaning, including also all straight and branched chain isomers of pentyl and hexyl. Preferred alkyl radicals for use in the present invention are C₁-C₆alkyl , including methyl, ethyl, n-propyl, isopropyl, n-butyl, isobutyl, see-butyl, tert-butyl, n-pentyl and n-hexyl, especially C₁-C₄alkyl such as methyl, ethyl, n-propyl, isopropyl, t-butyl, n-butyl and isobutyl. Methyl and isopropyl are typically preferred. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene—O-alkyl, alkylthio, —NH₂, —NH(alkyl), —N(alkyl)₂, —NH(cycloalkyl), —O—C(═O)-alkyl, —O—C(═O)-aryl, —O—C(═O)-cycloalkyl, —C(═O)OH and —C(═O)O-alkyl. It is generally preferred that the alkyl group is unsubstituted, unless otherwise indicated.

“C₂-Cₙalkenyl” represents a straight or branched aliphatic hydrocarbon radical containing at least one carbon-carbon double bond and having the number of carbon atoms designated, e.g. C₂-C₄alkenyl means an alkenyl radical having from 2 to 4 carbon atoms; C₂-C₆alkenyl means an alkenyl radical having from 2 to 6 carbon atoms. Non-limiting alkenyl groups include ethenyl, propenyl, n-butenyl, 3-methylbut-2-enyl, n-pentenyl and hexenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene—O-alkyl, alkylthio, —NH₂, —NH(alkyl), —N(alkyl)₂, —NH(cycloalkyl), —O—C(═O)-alkyl, —O—C(═O)-aryl, —O—C(═O)-cycloalkyl, —C(═O)OH and —C(═O)O-alkyl. It is generally preferred that the alkenyl group is unsubstituted, unless otherwise indicated.

“C₂-Cₙalkynyl” represents a straight or branched aliphatic hydrocarbon radical containing at least one carbon-carbon triple bond and having the number of carbon atoms designated, e.g. C₂-C₄alkynyl means an alkynyl radical having from 2 to 4 carbon atoms; C₂-C₆alkynyl means an alkynyl radical having from 2 to 6 carbon atoms. Non-limiting alkenyl groups include ethynyl, propynyl, 2-butynyl and 3-methylbutynyl pentynyl and hexynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene—O-alkyl, alkylthio, —NH₂, —NH(alkyl), —N(alkyl)2, —NH(cycloalkyl), —O—C(O)-alkyl, —O—C(O)-aryl, —O—C(O)-cycloalkyl, —C(O)OH and —C(O)O-alkyl. It is generally preferred that the alkynyl group is unsubstituted, unless otherwise indicated.

The term “Cₘ-Cₙhaloalkyl” as used herein represents Cₘ-Cₙalkyl wherein at least one C atom is substituted with a halogen (e.g. the Cₘ-Cₙhaloalkyl group may contain one to three halogen atoms), preferably iodine, bromine, or fluorine. Typical haloalkyl groups are C₁-C₂haloalkyl, in which halo suitably represents iodo. Exemplary haloalkyl groups include iodomethyl, diiodomethyl and triiodomethyl. As used herein, only one of the halogens can be radioactive.

The term “Cₘ-Cₙhydroxyalkyl” as used herein represents Cₘ-Cₙalkyl wherein at least one C atom is substituted with one hydroxy group. Typical Cₘ-Cₙhydroxyalkyl groups are Cₘ-Cₙalkyl wherein one C atom is substituted with one hydroxy group. Exemplary hydroxyalkyl groups include hydroxymethyl and hydroxyethyl.

The term “Cₘ-Cₙalkylene” as used herein represents a straight or branched bivalent alkyl radical having the number of carbon atoms indicated. Preferred Cₘ-Cₙalkylene radicals for use in the present invention are C₁-C₃alkylene. Non-limiting examples of alkylene groups include —CH₂—, —CH₂CH₂—, —CH₂CH₂CH₂—, —CH(CH₃)CH₂CH₂—, —CH(CH₃)— and —CH(CH(CH₃)₂)—.

“Cₘ-Cₙalkoxy” represents a radical Cₘ-Cₙalkyl—O— wherein Cₘ-Cₙalkyl is as defined above. Of particular interest is C₁-C₄alkoxy which includes methoxy, ethoxy, n-propoxy, isopropoxy, t-butoxy, n-butoxy, see-butoxy and isobutoxy. Methoxy and isopropoxy are typically preferred. C₁-C₆alkoxy has a corresponding meaning, expanded to include all straight and branched chain isomers of pentoxy and hexoxy.

The term “Me” means methyl, and “MeO” means methoxy. The term “amino” represents the radical —NH₂. The term “halo” represents a halogen radical such as fluoro, chloro, bromo, iodo, or astato. Typically, halo groups are iodo, bromo or astato. The term “aryl” represents an aromatic ring, for example a phenyl, biphenyl or naphthyl group.

The term “heterocycloalkyl” represents a stable saturated monocyclic 3-12 membered ring containing 1-4 heteroatoms independently selected from O, S and N. In one embodiment the stable saturated monocyclic 3-12 membered ring contains 4 N heteroatoms. In a second embodiment the stable saturated monocyclic 3-12 membered ring contains 2 heteroatoms independently selected from O, S and N. In a third embodiment the stable saturated monocyclic 3-12 membered ring contains 3 heteroatoms independently selected from O, S and N. A heterocycloalkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene—O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. It is generally preferred that the heterocycloalkyl group is unsubstituted, unless otherwise indicated.

The term “heteroaryl” represents a stable aromatic ring containing 1-4 heteroatoms independently selected from O, S and N. In preferred embodiments, heteroaryl moieties useful in the present disclosure have 6 ring atoms. In one embodiment of the invention the stable aromatic ring system contains one heteroatom that is N.

The term “aminoCₘ-Cₙalkyl” represents a Cₘ-Cₙalkyl radical as defined above which is substituted with an amino group, i.e. one hydrogen atom of the alkyl moiety is replaced by an NH₂-group. Typically, “aminoCₘ-Cₙalkyl” is aminoC₁-C₆alkyl.

The term “aminoCₘ-Cₙalkylcarbonyl” represents a Cₘ-Cₙalkylcarbonyl radical as defined above, wherein one hydrogen atom of the alkyl moiety is replaced by an NH₂-group. Typically, “aminoCₘ-Cₙalkylcarbonyl” is aminoC₁-C₆alkylcarbonyl. Examples of aminoCₘ-Cₙalkylcarbonyl include but are not limited to glycyl: C(═O)CH₂NH₂, alanyl: C(═O)CH(NH₂)CH₃, valinyl: C═OCH(NH₂)CH(CH₃)₂, leucinyl: C(═O)CH(NH₂)(CH₂)₃CH₃, isoleucinyl: C(═O)CH(NH₂)CH(CH₃)(CH₂CH₃) and norleucinyl: C(═O)CH(NH₂)(CH₂)₃CH₃ and the like. This definition is not limited to naturally occurring amino acids.

Related terms, are to be interpreted accordingly in line with the definitions provided above and the common usage in the technical field.

As used herein, the term “(═O)” forms a carbonyl moiety when attached to a carbon atom. It should be noted that an atom can only carry an oxo group when the valency of that atom so permits. The term “monophosphate, diphosphate and triphosphate ester” refers to groups:

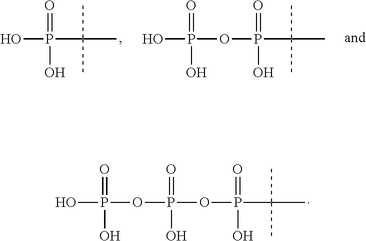

The term “thio-monophosphate, thio-diphosphate and thio-triphosphate ester” refers to groups:

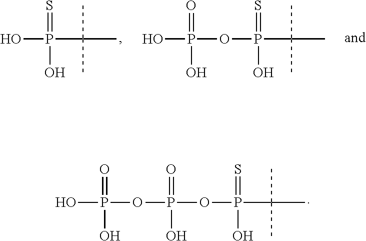

As used herein, the radical positions on any molecular moiety used in the definitions may be anywhere on such a moiety as long as it is chemically stable. When any variable present occurs more than once in any moiety, each definition is independent.

Whenever used herein, the phrases “compounds of Formula 1”, “compounds of Formula 1A,” “compounds of Formula 2” or “the compounds of the invention” or similar phrases, are meant to include the compounds of Formula 1 and subgroups of compounds of Formula 1, the compounds of Formula 2 and subgroups of compounds of Formula 2, including the possible stereochemically isomeric forms, and their pharmaceutically acceptable salts and solvates.

The term “solvates” covers any pharmaceutically acceptable solvates that the compounds of Formula 1, and 2, as well as the salts thereof, are able to form. Such solvates are, for example, hydrates, alcoholates, e.g., ethanolates, propanolates, and the like, especially hydrates.

In general, the names of compounds used in this application are generated using ChemDraw Professional 16.0. In addition, if the stereochemistry of a structure or a portion of a structure is not indicated with for example bold or dashed lines, the structure or portion of that structure is to be interpreted as encompassing all stereoisomers of it.

Linkers may also be selected to facilitate bonding of the respective moieties to the core structure. For example, as discussed in greater details below with respect to a preferred synthesis pathway for the prosthetic compound, a representative linker is a bifunctional alkyl chain (e.g., —CH₂—, —C₂H₄—, —C₃H₆—, etc.) having from 1 to 6 carbon atoms, in which one carbon atom may be substituted with a cyclic (hydrocarbon ring) radical or heterocyclic (heterocyclic ring) radical. Representative heterocyclic radicals have at least one nitrogen atom in the heterocyclic ring. Specific examples of such heterocyclic radicals are therefore diazinyl, diazolyl, triazinyl, triazolyl, tetrazinyl, and tetrazolyl radicals. These and other heterocyclic radicals, or otherwise cyclic radicals, may optionally be fused to a another cyclic or heterocyclic radical, or otherwise fused to a another cyclic or heterocyclic radical that is itself part of a fused ring system (e.g., a triazolyl radical may be fused to an 8-membered cyclic or heterocyclic radical that is itself fused to two 6-membered cyclic rings, as in the case of the triazolyl radical (or other nitrogen atom-substituted heterocyclic hydrocarbon radical) being fused to a dibenzoazocanyl radical). Therefore, linkers containing three or more fused rings, such as hydrocarbon rings, heterocyclic rings, and combinations of these rings, are possible. A representative charged group linker, L₂, is a bivalent substituted or unsubstituted alkyl chain having from 1 to 6 carbon atoms, a substituted or unsubstituted alkenyl chain, or a substituted or unsubstituted alkynyl chain. Generally, L₁, L₂, L₃ and/or La may be (or may comprise) substituted or unsubstituted bivalent alkyl radicals, having from 1 to 6 carbon atoms, wherein one or more carbon atoms may be substituted with and/or replaced by a heteroatom such as NH, O, or S, or otherwise may be substituted with or replaced by another alkyl radical (e.g., resulting in the formation of a branched alkyl radical) having from 1 to 8 carbon atoms that may be linear, branched, or cyclic. For example, one carbon atom of an alkyl radical may be substituted to provide a carbonyl (C═O) group, and an adjacent carbon atom replaced by NH, thereby resulting in a peptide/amide linkage —(C═O)—NH—. Representative linkers L₁, L₂, L₃, and L₄ can therefore include divalent alkyl radicals having one or more of such peptide linkages, —NH-linkages, —(C═O)-linkages, and/or cyclic —C₆H₄— linkages, including combinations of any two, three, or four of such linkages, incorporated into the alkyl chain. In addition, in the case of bivalent alkyl radicals for L₁, L₂, and/or L₃, a carbon-carbon double bond and/or a carbon-carbon triple bond may be formed between one or more pairs of adjacent carbon atoms, to provide bivalent, unsaturated (e.g., olefinic) alkyl radicals.

The selection of an appropriate labeling method for a biomolecule requires careful consideration of the fate of the molecule after its interaction with the biological milieu. For radioiodinated proteins and peptides, circumventing the action of deiodinases such as those normally involved in thyroid hormone metabolism is an important concern. Reagents such as N-succinimidyl 3-[¹³¹I] iodobenzoate (SIB) yield proteins that do not undergo appreciable deiodination in vivo based on the tyrosine-dissimilar structure of the site where the radiolabel resides. However, when a labeled protein or peptide undergoes cellular internalization after binding to a cell surface receptor or antigen, then, depending on its intercellular routing, considerable loss of label from the targeted cell can occur even with SIB labeling.

In some embodiments, the targeted radiotherapy methods of the invention can utilize radiohalogens that emit radiations with ranges in tissue of less than 15 mm. These include alpha emitters such as ²¹¹At, beta emitters such as ¹³¹I and Auger electron emitters such as, ⁷⁷Br, ¹²³I, and ¹²⁵I, and the like. Diagnostic imaging methods of the invention utilize radiations with ranges in tissue greater than 5 mm such that the radiation can be detected outside the body by positron emission tomography (PET) utilizing radiohalogens such as ⁷⁵Br, ⁷⁶Br, ¹²⁴I and the like; single photon emission computed tomography (SPECT) utilizing radiohalogens such as ¹²³I, ¹³¹I, and ⁷⁷Br and the like; or intra-operative imaging that can be performed with any of the radiohalogens indicated above. See U.S. Pat. No. 5,302,700, herein incorporated by reference. In particular, ¹³¹I emits low energy β-particles with a maximum tissue range of 2.3 mm. Stein et al. (2003) Cancer Res 63:111-118, herein incorporated by reference. Theranostic methods of the invention utilize either 1) the same radiohalogen to perform targeted radiotherapy and diagnostic imaging (for example, ¹³¹I, ¹²³I, ⁷⁷Br and the like) or 2) different radiohalogens of the same element to perform targeted radiotherapy and diagnostic imaging (for example, ¹²⁴I and ¹³¹I; ¹²³I and ¹³¹I; ⁷⁷Br and ⁷⁶Br; ⁷⁷Br and ⁷⁵Br; and the like). In some embodiments (e.g., employing Formula 2 compounds), other radiometals can be used, which bind to the metal chelate portion of the molecule.

Representative biomolecules that may be coupled to radiolabeled prosthetic compounds described above include any molecule that specifically binds to a cell surface receptor, antigen or transporter. Representative cell surface antigens or receptors include those that are internalized by the cell. Biomolecules can be internalized by the cell over seconds, minutes, hours, or days. Preferred biomolecules are internalized rapidly, i.e., most of the biomolecule is internalized after minutes to hours. A biomolecule is considered to bind specifically when it binds with an affinity constant (K_{D}) of 10⁻⁶ M or less, preferably 10⁻⁸ M⁻¹ or less.

A biomolecule can be an antibody, a fragment of an antibody, or a synthetic peptide that binds specifically to a cell surface antigen, receptor or transporter. Antibodies include monoclonal antibodies (mAbs) and antibody fragments include VHH molecules (also known as single-domain antibody fragments (sdAbs) or nanobodies). In a preferred embodiment, the biomolecule is an internalizing antibody or antibody fragment. Any antibody that specifically binds to a cell surface antigen and is internalized by the cell is an internalizing antibody. The antibody can be an immunoglobulin of any class, i.e., IgG, IgA, IgD, IgE, or IgM, and can be obtained by immunization of a mammal such as a mouse, rat, rabbit, goat, sheep, primate, human or other suitable species, including those of the Camelidae family. The antibody can be polyclonal, i.e., obtained from the serum of an animal immunized with a cell surface antigen or fragment thereof. The antibody can also be monoclonal, i.e., formed by immunization of a mammal using the cell membrane or surface ligand or antigen or a fragment thereof, fusion of lymph or spleen cells from the immunized mammal with a myeloma cell line, and isolation of specific hybridoma clone, as is known in the art. The antibody can also be a recombinant antibody, e.g., a chimeric or interspecies antibody produced by recombinant DNA methods. A preferred internalizing antibody is a humanized antibody comprising human immunoglobulin constant regions together with murine variable regions which possess specificity for binding to a cell surface antigen (see, e.g., Reist et al., 1997). If a fragment of an antibody is used, the fragment should be capable of specific binding to a cell surface antigen. The fragment can comprise, for example, at least a portion of an immunoglobulin light chain variable region and at least a portion of an immunoglobulin heavy chain variable region. A biomolecule can also be a synthetic polypeptide which specifically binds to a cell surface antigen. For example, the biomolecule can be a synthetic polypeptide comprising at least a portion of an immunoglobulin light chain variable region and at least a portion of an immunoglobulin heavy chain variable region, as described in U.S. Pat. No. 5,260,203 or as otherwise known in the art.

Many of the known molecular targets for labeled mAbs are internalizing antigens and receptors. B-cell lymphoma (Press et al., 1994; Hansen et al., 1996), T-cell leukemia (Geissler et al., 1991) and neuroblastoma cells (Novak-Hofer et al., 1994) all possess antigens that are internalized rapidly. Internalizing receptors have been used to target mAbs to tumors. These include wild-type epidermal growth factor receptor (EGFR; gliomas and squamous cell carcinoma; Brady et al., 1992; Baselga et al., 1994), the p185 c-erbB-2 oncogene product, HER2 (breast and ovarian carcinomas; De Santes et al. 1992; Xu et al., 1997), and the transferrin receptor (gliomas and other tumors; Laske et al., 1997). Indeed, it has been suggested that internalization can occur with virtually any mAb that binds to a cell-surface antigen (Mattes et al., 1994; Sharkey et al., 1997a).

An advantage of mAb internalization for radioimmunotherapy is the potential for increasing the radiation absorbed dose delivered to the cell nucleus provided that the radioactivity is trapped on the targeted cell for a prolonged period. Radiation dosimetry calculations suggest that even with the multicellular range β-emitter ¹³¹I, shifting the site of decay from the cell membrane to cytoplasmic vesicles could increase the radiation dose received by the cell nucleus by a factor of two (Daghighian et al., 1996), thereby potentially increasing treatment. On the other hand, a disadvantage of mAb internalization is that this event exposes the mAb to additional catabolic processes that can result in the release of radioactivity from the tumor cell, decrease the radiation dose to cancer cells and increasing the radiation dose to normal tissues in the body.

Antigens or receptors that are internalized by the cell can eventually become localized within endosomes or lysosomes. The targeting moiety or internalization moiety are moieties that bind to the targeted diseased cells, such as cancer cells, and are internalized after binding to a cell surface receptor, a transporter, antigens found on the cell surface such as, for example, transmembrane receptors, extracellular growth factors, etc. In this manner, the compounds of the invention can be directed to any population of diseased cells or tumor cells. Thus, it can be broadly used to target any cancer, tumor, or malignant growth. The compounds of the invention can be targeted to human epidermal growth factor receptor 2 (HER2), epidermal growth factor receptor (EGFR), its tumor-specific mutant EGFRvIII, vascular endothelial growth factor (VEGF), VEGFA/B, EGFR (HER1/ERBB1), HER2 (ERBB2/neu), ALK, Ax1, CD20, CD30, CD38, CD47, CD52, CDK4, CDK6, PD-1, PD-L1, KIT, VEGFR1/2/3, BAFF, HDAC, Proteasome, ABL, FLT3, KIT, MET, RET, IL-6, IL-6R, IL-1B, EGFR (HER1/ERBB1), MEK, ROS1, BRAF, ABL, RANKL, B4GALNT1(GD2), SLAMF7, (CS1/CD319/CRACC), mTOR, BTK, P13Kδ, PDGFR, PDGFRα, PDGFRβ, CTLA4, PARP, HDAC, FGFR1-3, RAF, RET, JAK1/2, JAK3, Smoothened, MEK, BCL2, PTCH, PIGF, EMP2, CSF-1R, LYPD3, and the like. See, for example, Abramson, R. (2017) Overview for Targeted Therapies for Cancer, My Cancer Genome, found on the world-wide web at the “mycancergenome” website in the overview-of-targeted-therapies-for-cancer section.

In some embodiments, the targeting moiety can be selected from anti-HER2 VHH sequences such as those set forth in SEQ ID NOS: 1-5 and fragments and variants thereof that retain the binding specificity of the sequences. That is, the invention encompasses fragments, analogs, mutants, variants, and derivatives of the radiolabeled VHH domains. These oligoclonal VHHs are able to target a range of different epitopes on the HER2 receptor. Some of the VHHs do not compete with trastuzumab for binding on HER2. In some embodiments, the fragment, analog, mutant, variant and/or derivative of the VHH sequences provided herein has at least 80%, 85%, 90%, 95%, 96%, 97%, 98%, or 99% sequence identity with at least one of SEQ ID NOS: 1-5. See Table 1.

In determining the degree of sequence identity between two amino acid sequences, one of skill in the art may take into account “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Amino acid sequences and nucleic acid sequences are exactly the same if they have 100% sequence identity over their entire length.

As used herein, where a sequence is defined as being “at least X % identical” to a reference sequence, e.g., “a polypeptide at least 95% identical to SEQ ID NO:2,” it is to be understood that “X % identical” refers to absolute percent identity, unless otherwise indicated. The term “absolute percent identity” refers to a percentage of sequence identity determined by scoring identical amino acids or nucleic acid as one and any substitution as zero, regardless of the similarity of mismatched amino acids or nucleic acids. In a typical sequence alignment, the “absolute percent identity” of two sequences is presented as a percentage of amino acid or nucleic acid “identities”. In cases where an optimal alignment of two sequences requires the insertion of a gap in one or both of the sequences, an amino acid residue in one sequence that aligns with a gap in the other sequences is counted as a mismatch for purposes of determining percent identity. Gaps can be internal or external, i.e., a truncation. Absolute percent identity can be readily determined using, for example, the Clustal W program, version 1.8, June 1999, using default parameters (Thompson et al. (1994) Nucleic Acids Res 22:4673-4680).

As indicated, the radiolabeled biomolecules of the invention can be targeted to any diseased or malignant cell population. In some instances, it may be preferred to use small biomolecules. Brain metastases are cancer cells that have spread to the brain from primary tumors in other organs in the body. Metastatic tumors are among the most common mass lesions in the brain. An estimated 24-45% of all cancer patients have brain metastases, Lung, breast, melanoma, colon, and kidney cancers commonly spread to the brain. Brain metastases are associated with poor survival and high morbidity. Improving therapies for metastatic brain tumors is an aspect of the present invention.

The calculated pore size of a brain metastasis of breast cancer is less than 10 nm in diameter. (Mittapali et al. (2017) Cancer Res 77(2): 238-246). Therefore, small molecules are needed to effectively target and treat metastatic brain tumors. For use in the diagnosis and treatment of metastatic brain tumors, the targeting biomolecules of the invention are small molecules, including, but not limited to, affibodies, designed ankyrin repeat proteins (DARPins), aptamers, and VHH molecules (also known as single domain antibody fragments (sdAb) or nanobodies), collectively called small biomolecules herein. Other “small molecule” scaffolds are characterized by mass/size, e.g., less than 10 nm in size or less than 25 kDa. As indicated, these small biomolecules are designed to bind to a portion of the cancer cells. For example, VHHs can be prepared to specifically bind receptors on the cancer cells, such as human epidermal growth factor receptor-2 (HER2) or any of the other receptors listed above. See, for example, U.S. Pat. Nos: 9,234,028; 9,309,515; 8,524,244; 9,234,065; Liu et al. (2012) J Transl. Med. 10:148; Gijs et al. (2016) Pharmaceuticals (Basel) 9(2):29; Moosavian et al. (2015) Iran J. Basic Med. Sci. 18(6): 576-586; Mahlknecht et al. (2012) Proc. Natl. Acad. Sci. 110:8170-8175;

Due to their small size, VHHs, aptamers and other small biomolecules diffuse and distribute efficiently throughout solid tumors, and due to their high binding specificity and affinity to their target antigens, high tumor uptake of the small biomolecules can be observed. Importantly, their half-life in the bloodstream is significantly shorter than full-length antibodies or larger targeting proteins, allowing rapid clearance of the unbound fraction of the small biomolecule by the kidneys, leading to higher tumor-to-normal tissue ratios shortly after their administration. VHHs are easily generated in nanomolar to picomolar affinity by cloning from immunized camels or llamas and selection by phage display panning. Moreover, VHHs or sdAb are stable and easily produced in large quantities using industrial grade methods and qualified bacteria, yeast, or mammalian cells. Compared with other small protein-based targeting vectors, VHHs generally offer significant advantages in terms of stability, solubility, expression yields, construction of multimers, as well as the ability to recognize hidden or uncommon epitopes. See, U.S. Pat. Nos: 6,248,516; 6,300,064; 6,846,634; 6,846,634; 6,696,245; 9,243,065; 7,696,320; all of which are herein incorporated by reference.

Aptamers are oligonucleotide or peptide molecules that bind to a specific target molecule. Aptamers can be nucleic acid molecules (DNA, RNA, XNA) and consist of short strands of oligonucleotides, peptide molecules that consist of one or more short variable peptide domains. Aptamers offer molecular recognition properties readily produced by chemical synthesis, possess desirable storage properties, and elicit little or no immunogenicity in therapeutic applications. See, Keefe et al. (2010) Nature Reviews Drug Discovery 9:537-550; Ellington and Szostak (1990) Nature 346:818-822; Tuerk and Gold (1990) Science 249:505-510; Kulbachinskiy, A. V. (2007) Biochemistry 72:1505-1518; all of which are herein incorporated by reference.

The ‘(calculated mean) effective dose’ of radiation within a subject as used herein refers to the tissue-weighted sum of the equivalent doses in all specified tissues and organs of the body. It takes into account the type of radiation and the nature of each organ or tissue being irradiated. It is the central quantity for dose limitation in radiological protection in the international system of radiological protection devised by the International Commission on Radiological Protection (ICRP). The SI unit for effective dose is the Sievert (Sv) which is one joule/kilogram (J/kg). The effective dose replaced the former “effective dose equivalent” in 1991 in the ICRP system of dose quantities. For procedures using radiopharmaceuticals, the effective dose is typically expressed per unit of injected activity, i.e. expressed in mSv/MBq. The effective dose for the individual patient will then depend upon the injected activity of the radiopharmaceutical, expressed in MBq, and the calculated mean effective dose, expressed in mSv/MBq.

The effective dose for radiopharmaceuticals is calculated using OLINDA/EXM® software that was approved in 2004 by the FDA. The OLINDA/EXM® personal computer code performs dose calculations and kinetic modeling for radiopharmaceuticals (OLINDA/EXM stands for Organ Level Internal Dose Assessment/Exponential Modeling). OLINDA® calculates radiation doses to different organs of the body from systemically administered radiopharmaceuticals and performs regression analysis on user-supplied biokinetic data to support such calculations for nuclear medicine drugs. These calculations are used to perform risk/benefit evaluations of the use of such pharmaceuticals in diagnostic and therapeutic applications in nuclear medicine. The technology employs several standard body models for adults, children, pregnant women and others, that are widely accepted and used in the internal dose community. The calculations are useful to pharmaceutical industry developers, nuclear medicine professionals, educators, regulators, researchers and others who study the accepted radiation doses that should be delivered when radioactive drugs are given to patients or research subjects.

The calculated effective dose depends on the chosen standard body model and the chosen voiding bladder model. The values provided herein have been calculated using the female adult model and a voiding bladder interval of 1 h.

Thus, in certain embodiments, the prevention and/or treatment of cancer is achieved by administering a radiolabeled small biomolecule, i.e., an aptamer, VHH or functional fragments thereof, and the like, as disclosed herein to a subject in need thereof, characterized in that the small biomolecule has a calculated mean effective dose of between 0.001 and 0.05 mSv/MBq in a subject, such as but not limited to a calculated mean effective dose of between 0.02 and 0.05 mSv/MBq, more preferably between 0.02 and 0.04 mSv/MBq, most preferably between 0.03 and 0.05 mSv/MBq.

Accordingly, the dose of radioactivity applied to the patient per administration must be high enough to be effective but must be below that which would result in dose limiting toxicity (DLT). For pharmaceutical compositions comprising radiolabeled antibodies, e.g. with ¹³¹Iodine, the maximally tolerated dose (MTD) must be determined which must not be exceeded in therapeutic settings.

The proteins and peptides (collectively referred to as biomolecules below) as envisaged herein and/or the compositions comprising the same are administered according to a regimen of treatment that is suitable for preventing and/or treating the disease or disorder to be prevented or treated. The clinician will generally be able to determine a suitable treatment regimen. Generally, the treatment regimen will comprise the administration of one or more small biomolecules, such as VHH sequences or polypeptides, or of one or more compositions comprising the same, in one or more pharmaceutically effective amounts or doses.

The desired dose may conveniently be presented in a single dose or as divided doses (which can again be sub-dosed) administered at appropriate intervals. An administration regimen could include long-term (i.e., at least two weeks, and for example several months or years) or daily treatment. In particular, an administration regimen can vary between once a day to once a year, such as between once a day and once every twelve months, such as but not limited to once a week. Thus, depending on the desired duration and effectiveness of the treatment, pharmaceutical small biomolecule compositions as disclosed herein may be administered once or several times, also intermittently, for instance daily for several days, weeks or months and in different dosages. The amount applied of the small biomolecule compositions disclosed herein depends on the nature of the cancer or other disease to be treated. Multiple administrations may be preferred in order to achieve effective radiation dose delivery to the cancer while avoiding DLT. However, radiolabeled materials are typically administered at intervals of 4 to 24 weeks apart, preferably 8 to 20weeks apart. The skilled artisan knows how to divide the administration into two or more applications, which may be applied shortly after each other, or at some other predetermined interval ranging e.g. from 1day to 1 week.

In particular, the biomolecules disclosed herein may be used in combination with other pharmaceutically active compounds or principles that are or can be used for the prevention and/or treatment of the diseases and disorders cited herein, as a result of which a synergistic effect may or may not be obtained. Examples of such compounds and principles, as well as routes, methods and pharmaceutical formulations or compositions for administering them will be clear to the clinician.

In the context of this invention, “in combination with”, “in combination therapy” or “in combination treatment” shall mean that the radiolabeled biomolecule, for example VHH, aptamer, and the like, as disclosed herein are applied together with one or more other pharmaceutically active compounds or principles to the patient in a regimen wherein the patient may profit from the beneficial effect of such a combination. In particular, both treatments are applied to the patient in temporal proximity. In a preferred embodiment, both treatments are applied to the patient within four weeks (28 days). More preferably, both treatments are applied within two weeks (14 days), more preferred within one week (7 days). In a preferred embodiment, the two treatments are applied within two or three days. In another preferred embodiment, the two treatments are applied at the same day, i.e. within 24 hours. In another embodiment, the two treatments are applied within four hours, or two hours, or within one hour. In another embodiment, the two treatments are applied in parallel, i.e. at the same time, or the two administrations are overlapping in time.

In particular non-limiting embodiments, the radiolabeled biomolecules of the invention are applied together with one or more therapeutic antibodies or therapeutic antibody fragments. Thus, in these particular non-limiting embodiments, the targeted radiotherapy with the radiolabeled biomolecule is combined with regular immunotherapy with one or more therapeutic antibodies or therapeutic antibody fragments. In further particular embodiments, the radiolabeled biomolecules are used in a combination therapy or a combination treatment method with one or more therapeutic antibodies or therapeutic antibody fragments, such as but not limited to a combination treatment with Trastuzumab (Herceptin®) and/or Pertuzumab (Perjeta®).

For example, the radiolabeled biomolecules and the one or more therapeutic antibodies or therapeutic antibody fragments, such as but not limited to Trastuzumab (Herceptin®) and/or Pertuzumab (Perjeta®), may be infused at the same time, or the infusions may be overlapping in time. If the two drugs are administered at the same time, they may be formulated together in one single pharmaceutical preparation, or they may be mixed together immediately before administration from two different pharmaceutical preparations, for example by dissolving or diluting into one single infusion solution. In another embodiment, the two drugs are administered separately, i.e., as two independent pharmaceutical compositions. In one preferred embodiment, administration of the two treatments is in a way that tumor cells within the body of the patient are exposed to effective amounts of the cytotoxic drug and the radiation at the same time. In another preferred embodiment, effective amounts of both the radiolabeled biomolecules of the invention and the one or more therapeutic antibodies or therapeutic antibody fragments, such as but not limited to Trastuzumab (Herceptin®) and/or Pertuzumab (Perjeta®) are present at the site of the tumor at the same time. The present invention also embraces the use of further agents, which are administered in addition to the combination as defined. This could be, for example, one or more further chemotherapeutic agent(s). It could also be one or more agent(s) applied to prevent, suppress, or ameliorate unwanted side effects of any of the other drugs given. For example, a cytokine stimulating proliferation of leukocytes may be applied to ameliorate the effects of leukopenia or neutropenia.

According to a further aspect, the use of the radiolabeled biomolecules as envisaged herein that specifically bind to a tumor-specific or cancer cell-specific target molecule of interest is provided for the preparation of a medicament for the prevention and/or treatment of at least one cancer-related disease and/or disorder in which said tumor-specific or cancer cell-specific target molecule is involved. Accordingly, the application provides biomolecules specifically binding to a tumor-specific or cancer cell-specific target, such as those set forth above, for use in the prevention and/or treatment of at least one cancer-related disease and/or disorder in which said tumor-specific or cancer cell-specific target is involved. In particular embodiments, methods for the prevention and/or treatment of at least one cancer-related disease and/or disorder are also provided, comprising administering to a subject in need thereof, a pharmaceutically active amount of one or more biomolecules including VHH sequences or functional fragments thereof, polypeptides, aptamers, etc., and/or pharmaceutical compositions as envisaged herein.

The subject or patient to be treated with the radiolabeled biomolecules described herein may be any warm-blooded animal, but is in particular, a mammal and more particularly, a human suffering from, or at risk of, a cancer-related disease and/or other disease disorder. The efficacy of the biomolecules, i.e., VHH sequences or functional fragments thereof, aptamers, polypeptides, and the like described herein, and of compositions comprising the same, can be tested using any suitable in vitro assay, cell-based assay, in vivo assay and/or animal model known per se, or any combination thereof, depending on the specific disease or disorder involved. Suitable assays and animal models will be clear to the skilled person.

Depending on the tumor-specific or cancer cell-specific target involved, the skilled person will generally be able to select a suitable in vitro assay, cellular assay or animal model to test the biomolecules described herein for binding to the tumor-specific or cancer cell-specific molecule; as well as for their therapeutic and/or prophylactic effect in respect of one or more cancer-related diseases and disorders.

Accordingly, biomolecules are provided comprising or essentially consisting of at least one radiolabeled biomolecule or functional fragments thereof for use as a medicament, and more particularly for use in a method for the treatment of a disease or disorder related cancer, including solid tumors.

In particular embodiments, the radiolabeled biomolecules envisaged herein are used to treat and/or prevent cancers and neoplastic conditions. Examples of cancers or neoplastic conditions include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma. Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, melanoma, neuroblastoma, retinoblastoma, leukemia, lymphoma, or Kaposi sarcoma. The biomolecules as envisaged herein can also be used to treat a variety of proliferative disorders. Examples of proliferative disorders include hematopoietic neoplastic disorders and cellular proliferative and/or differentiative disorders, such as but not limited to, epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, miscellaneous malignant neoplasms, gynecomastia carcinoma, bronchogenic carcinoma, including parancoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma), malignant mesothelioma, non-neoplastic polyps, adenomas, familial syndromes, colorectal carcinogenesis, colorectal carcinoma, carcinoid tumors, nodular hyperplasias, adenomas, and malignant tumors, including primary carcinoma of the liver and metastatic tumors, tumors of coelomic epithelium, serous tumors, mucinous tumors, endometrioid tumors, clear cell adenocarcinoma, cystadenofibroma, Brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stromal tumors such as, granulosa-theca cell tumors, thecomafibromas, androblastomas, hill cell tumors, and gonadoblastoma; and metastatic tumors such as Krukenberg tumors.

Imaging of radioactivity after administration of the biomolecule labeled with the claimed prosthetic compounds can be performed by standard radiological methods, including, for example, scanning the body with a gamma camera (radioscintigraphy), single photon emission computed tomography (SPECT) and positron emission tomography (PET) (see, e.g., Bradwell et al., Immunology Today 6:163-170, 1985). For in vivo use, the labeled prosthetic compound, coupled to a biomolecule, should be given in either diagnostically or therapeutically acceptable amounts. A therapeutically acceptable amount is an amount which, when given in one or more dosages, produces the desired therapeutic effect, e.g., shrinkage of a tumor, with a level of toxicity acceptable for clinical treatment. Such an administered amount will cause sufficient radiation to absorb within tumor cells so as to damage these cells, for example by disrupting their DNA. Such an administered amount preferably should cause minimal damage to neighboring and distant healthy cells.

Both the dose of a particular composition and the means of administering the composition can be determined based on specific qualities of the composition, the condition, age, and weight of the patient, the progression of the particular disease being treated, and other relevant factors. If the composition contains antibodies, effective dosages of the composition are in the range of about 5 μg to about 50 μg/kg of patient body weight, about 50 μg to about 5 mg/kg, about 100 μg to about 500 μg/kg of patient body weight, and about 200 to about 250 μg/kg. A diagnostically acceptable amount of radioactivity is an amount which permits detection of radioactivity from the labeled biomolecule as required for diagnosis, with a level of toxicity acceptable for diagnosis. Various embodiments are provided herein below.

Embodiment 1: A compound represented by Formula I (including prosthetic compounds and radiohalogen precursors):

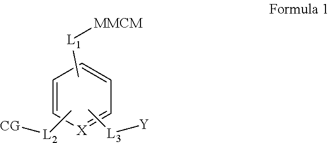

wherein:
X is CH or N;
L₁ and L₃ are independently selected from a bond, a substituted or unsubstituted alkyl chain, a substituted or unsubstituted alkenyl chain, a substituted or unsubstituted alkynyl chain, and a polyethylene glycol (PEG) chain;
MMCM is a macromolecule conjugating moiety;
L₂ is a substituted or unsubstituted alkyl chain, a substituted or unsubstituted alkenyl chain, a substituted or unsubstituted alkynyl chain, or a polyethylene glycol (PEG) chain comprising at least three oxygen atoms, wherein L₂ optionally contains a Brush Border enzyme-cleavable peptide;
CG is selected from guanidine, PO₃H, SO₃H, one or more charged D-amino acids, arginine or phosphono/sulfo phenylalanine, glutamate, aspartate, lysine, a hydrophilic carbohydrate moiety, a polyethylene glycol (PEG) chain, and guanidino-Z;
Z is (CH₂)ₙ;
n is greater than 1; and
Y is an alkyl metal radiohalogen precursor or a radioactive halogen selected from the group consisting of ¹⁸F, ⁷⁵Br, ⁷⁶Br, ⁷⁷Br, ¹²³I, ¹²⁴I, ¹²⁵I, ¹³¹I, and ²¹¹At, or a pharmaceutically acceptable salt or solvate thereof.

Embodiment 2: The compound of Embodiment 1, wherein Y is an alkyl metal radiohalogen precursor selected from the group consisting of trimethyl stannyl (SnMe₃), tri-n-butylstannyl (SnBu₃) and trimethylsilyl (SiMe₃).

Embodiment 3: The compound of Embodiment 1, wherein Y is a radioactive halogen selected from the group consisting of ⁷⁵Br, ⁷⁶Br, ⁷⁷Br, ¹²³I, ¹²⁴I, ¹²⁵I, ¹³¹I, and ²¹¹At.

Embodiment 4: The compound of any of Embodiment s 1-3, wherein MMCM is an active ester or (Gly) m, wherein m is 1 or more.

Embodiment 5: The compound of any one of Embodiments 1-3, wherein MMCM is selected from the group consisting of N-hydroxysuccinimide (NHS), tetrafluorophenol (TFP) ester, an isothiocyanate group, or a maleimide group.

Embodiment 6: The compound of any one of Embodiments 1-3, wherein MMCM is Gly-Gly-Gly.

Embodiment 7: The compound of any one of Embodiments 1-6, wherein L₂ is (CH₂)ₚ, wherein p=1 to 6.

Embodiment 8: The compound of any one of Embodiments 1-7, wherein the optional Brush Border enzyme-cleavable peptide is selected from the group consisting of Gly-Lys, Gly-Tyr and Gly-Phe-Lys.

Embodiment 9: The compound of any of Embodiments 1-8, represented by the following structure:

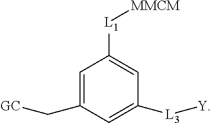

Embodiment 10: The compound of Embodiment 9, wherein the compound is N-succinimidyl 3-guanidinomethyl-5-[¹³¹I] iodobenzoate, or N-succinimidyl 3-[²¹¹At] astato-5-guanidinomethyl benzoate.

Embodiment 11: A radiolabeled biomolecule or intermediate, comprising the compound of any one of Embodiments 1-10 attached to a biomolecule.

Embodiment 12: The radiolabeled biomolecule or intermediate of Embodiment 11, wherein the biomolecule is selected from the group consisting of an antibody, an antibody fragment, a VHH molecule, an aptamer or variations thereof.

Embodiment 13: The radiolabeled biomolecule or intermediate of Embodiment 11 or 12, wherein said labeled biomolecule is a VHH.

Embodiment 14: The radiolabeled biomolecule or intermediate of Embodiment 13, wherein said VHH targets HER2.

Embodiment 15: The radiolabeled biomolecule or intermediate of Embodiment 14, wherein said VHH comprises an amino acid sequence selected from the sequences set forth in SEQ ID NOs: 1-5.

Embodiment 16: A pharmaceutical composition comprising the radiolabeled biomolecule of any of Embodiments 11-15 (where the compound is in the form of a prosthetic compound) in association with a pharmaceutically acceptable adjuvant, diluent or carrier.

Embodiment 17: A compound represented by Formula 2 (including prosthetic compounds and radiohalogen precursors):

MC—Cm—L₄—Cm—T Formula 2,
wherein:
MC is a polydentate metal chelating moiety;
Cm is thiourea, amide, or thioether;
L₄ is selected from a bond, a substituted or unsubstituted alkyl chain, a substituted or unsubstituted alkenyl chain, a substituted or unsubstituted alkynyl chain, optionally having NH, CO, or S on one or both termini, and a polyethylene glycol (PEG) chain; and
T is the compound of any of Embodiments 1-10,
or a pharmaceutically acceptable salt or solvate thereof.

Embodiment 18: The compound of Embodiment 17, wherein MC is a macrocyclic structure.

Embodiment 19: The compound of Embodiment 17, wherein MC is selected from DOTA, TETA, NOTP, and NOTA.

Embodiment 20: The compound of Embodiment 17, wherein MC is an acyclic polydentate ligand.

Embodiment 21: The compound of Embodiment 17, wherein MC is selected from EDTA, EDTMP, and DTPA.

Embodiment 22: The compound of any one of Embodiments 17-21, further comprising a metal associated with the MC.

Embodiment 23: The compound of Embodiment 21, wherein the metal is a radioactive metal selected from the group consisting of ¹⁷⁷Lu, ⁶⁴Cu, ¹¹¹In, ⁹⁰Y, ²²⁵Ac, ²¹³Bi, ²¹²Pb, ²¹²Bi, ⁶⁷Ga, ⁶⁸Ga, ⁸⁹Zr, and ²²⁷Th.

Embodiment 24: The compound of any one of Embodiments 17-23, wherein Y is an alkyl metal moiety (and the compound is a radiohalogen precursor).

Embodiment 25: The compound of Embodiment 24, wherein the alkyl metal moiety is selected from the group consisting of trimethyl stannyl (SnMe₃), tri-n-butylstannyl (SnBu₃) and trimethylsilyl (SiMe₃).

Embodiment 26: The compound of any one of Embodiments 17-23, wherein Y is a radioactive halogen, such as ⁷⁵Br, ⁷⁶Br, ⁷⁷Br, ¹²³I, ¹²⁴I, ¹²⁵I, ¹³¹I, or ²¹¹At (and the compound is a prosthetic compound).

Embodiment 27: A radiolabeled biomolecule or intemediate, comprising the compound of any one of Embodiments 17-26, attached to a biomolecule.

Embodiment 28: The radiolabeled biomolecule or intermediate of Embodiment 27, wherein the biomolecule is selected from the group consisting of an antibody, an antibody fragment, a VHH molecule and an aptamer.

Embodiment 29: The radiolabeled biomolecule or intermediate of Embodiment 27, wherein said labeled biomolecule is a VHH.

Embodiment 30: The radiolabeled biomolecule or intermediate of Embodiment 29, wherein said VHH targets HER2.

Embodiment 31: The radiolabeled biomolecule or intermediate of Embodiment 30, wherein said VHH comprises an amino acid sequence selected from the sequences set forth in SEQ ID NOs: 1-5.

Embodiment 32: A pharmaceutical composition comprising the radiolabeled biomolecule of any of Embodiments 27-31 (wherein the compound is a prosthetic compound), in association with a pharmaceutically acceptable adjuvant, diluent, or carrier.

Embodiment 33: A method of treatment for cancer comprising administering to an individual in need thereof an effective amount of the radiolabeled biomolecule of any one of Embodiments 11-15 or 27-31 or an effective amount of the pharmaceutical composition of claim or Embodiment 16 or 32.

The disclosure includes any combination of two, three, four, or more of the above-noted embodiments as well as combinations of any two, three, four, or more features or elements set forth in this disclosure, regardless of whether such features or elements are expressly combined in a specific embodiment herein.

The following examples are offered by way of illustration and not by way of limitation.

### Experimental

### Example 1: SIB-Arg

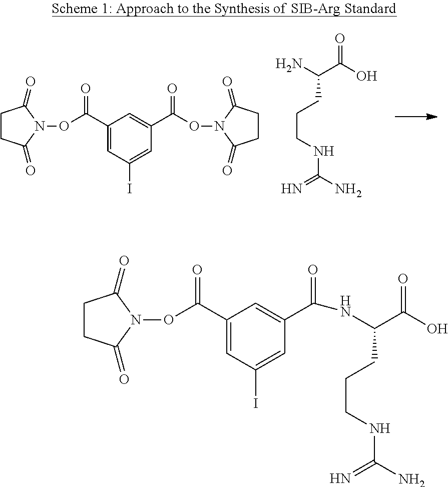 [SMILES: O=C(ON1C(=O)CCC1=O)c1cc(I)cc(C(=O)ON2C(=O)CCC2=O)c1 ; N=C(N)NCCC[C@H](N)C(=O)O ; N=C(N)NCCC[C@H](NC(=O)c1cc(I)cc(C(=O)ON2C(=O)CCC2=O)c1)C(=O)O]

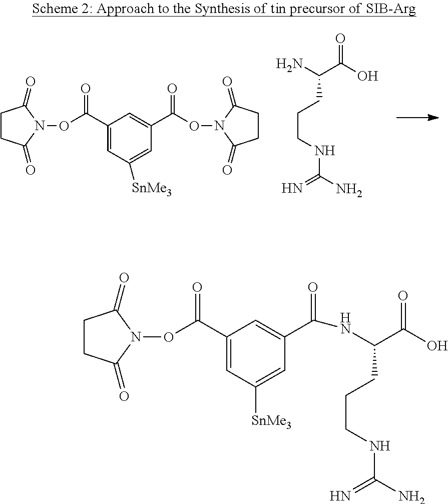

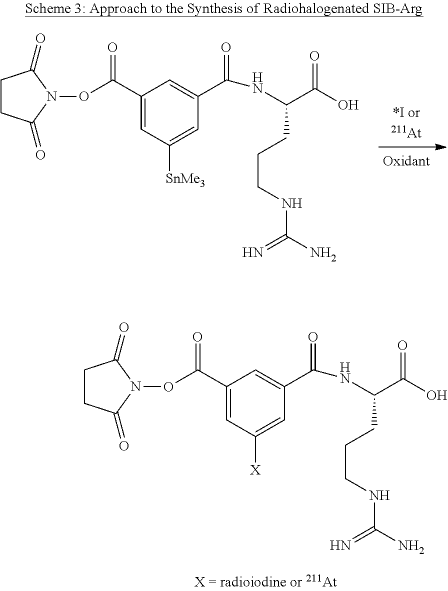

A solution of D-arginine in 0.1M sodium carbonate buffer, pH 8.5 (174.2 mg; 1 mmol in 3.5 ml) is gradually added to a solution of bis (2,5-dioxopyrrolidin-1-yl) 5-iodoisophthalate (486.2 mg; 1 mmol) in tetrahydrofuran (THF; 5.0 ml). The mixture is stirred at room temperature and the progress of the reaction is followed by thin layer chromatography (TLC). After the solvents are evaporated, the crude material is subjected to reversed-phase semi-preparative high-performance liquid chromatography (HPLC). Following the same procedure, 1 mmol (524 mg) of bis (2,5-dioxopyrrolidin-1-yl) 5-(trimethylstannyl) isophthalate is conjugated with 1 mmol of D-arginine. The tin precursor is radiohalogenated using standard conditions, purified and then conjugated to a macromolecule.

### Example 2: Arg-Gly-Tyr-PEG-SIB

A molecule containing the guanidine-bearing amino acid arginine, Brush Border enzyme-cleavable linker dipeptide GlyTyr, and connected to the SIB moiety via a PEG linker (Arg-Gly-Tyr-PEG-SIB), is shown below in Schemes 4-6. The radiolabeled version of this molecule, for example, Arg-Gly-Tyr-PEG-[¹³¹I]SIB, is obtained from the corresponding tin precursor using a standard iododestannylation reaction.

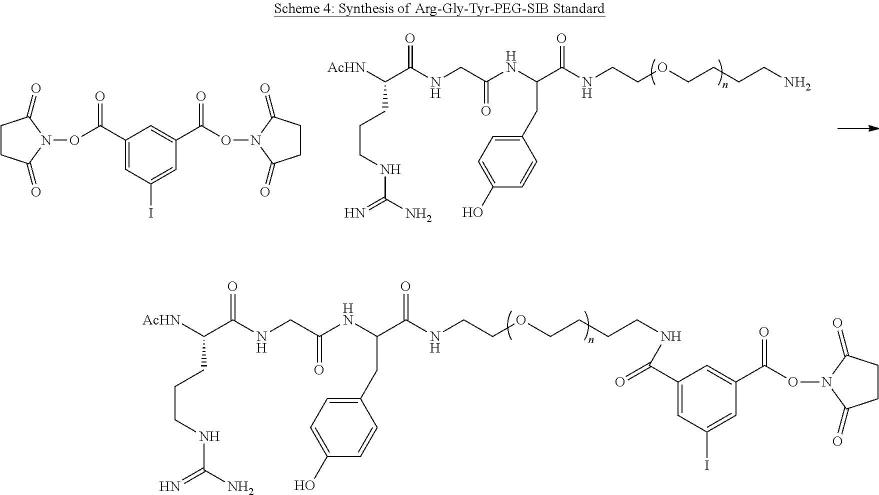

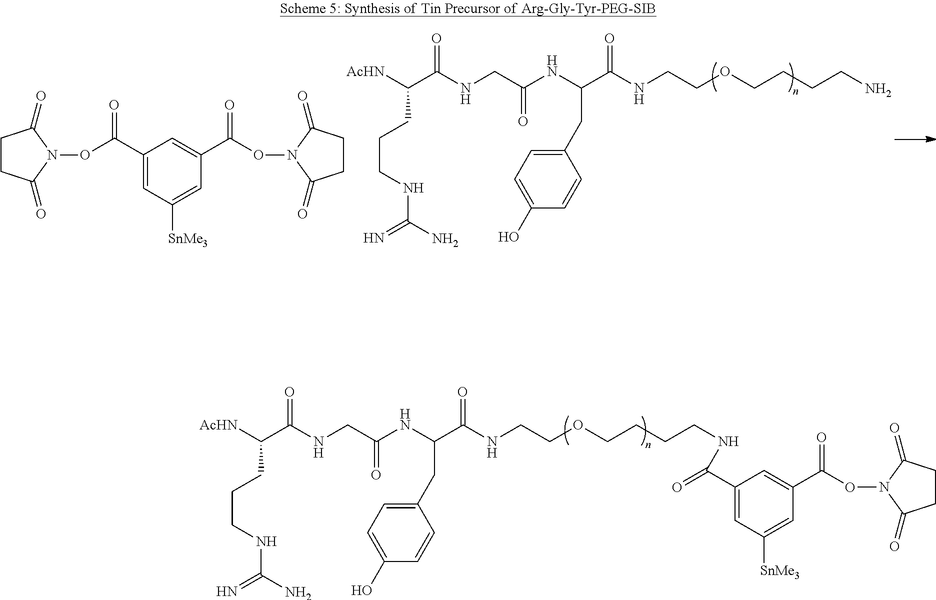

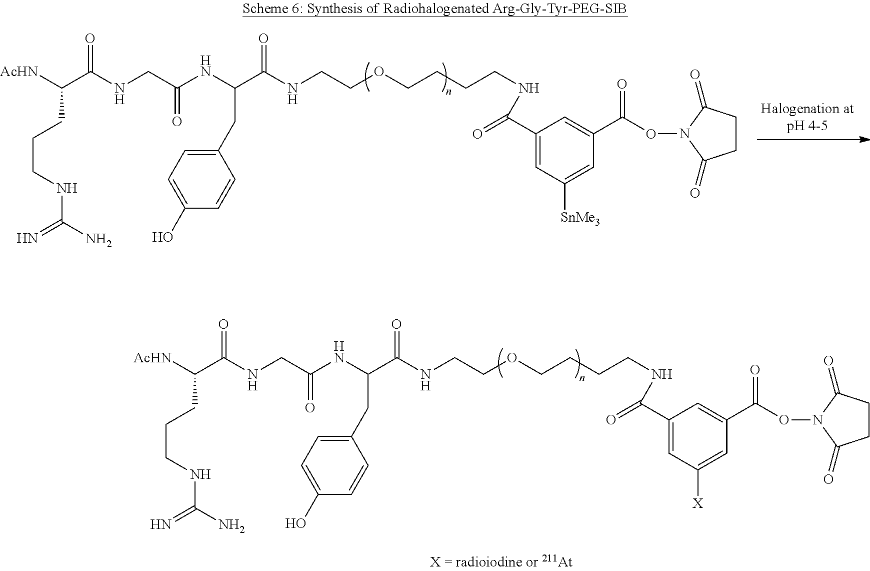

N-Acetyl argininyl-glycyl-tyrosine is synthesized by solid-phase peptide synthesis and is coupled to PEG diamine (n=2 to 4). Alternatively, PEG diamine can be anchored to a trityl chloride resin and the three amino acids can be attached sequentially. The resultant peptide derivative (1 mmol) is reacted with bis (2,5-dioxopyrrolidin-1-yl) 5-iodoisophthalate (486.2 mg; 1 mmol) in a mixture of THF and 0.1 M sodium carbonate buffer, pH 8.5. The progress of the reaction is followed by reversed-phase HPLC, and upon completion, the product is isolated by reversed-phase semi-preparative HPLC. The tin precursor is synthesized in a similar fashion by substituting bis (2,5-dioxopyrrolidin-1-yl) 5-(trimethylstannyl) isophthalate for bis (2,5-dioxopyrrolidin-1-yl) 5-iodoisophthalate. The tin precursor is radiohalogenated and purified for conjugation with a macromolecule using standard conditions.

### Example 3: DOTA-PEG-SIB

The scheme for the synthesis of DOTA-PEG-SIB is shown in Scheme 7. The same approach can be used to synthesize its tin precursor. The tin precursor can be labeled with radioiodine using standard conditions; the DOTA moiety present in both the iodo and tin derivatives can be complexed with nonradioactive lutetium. Unlike SIB-DOTA (Vaidyanathan et al. (2012) Bioorg. Med. Chem. 20(24):6929-6939), all four COOH groups in the DOTA macrocycle are available to complex with a metal ion and the PEG linker replaces the hydrophobic 6-carbon alkyl chain. Also, and importantly, the linker could include a Brush Border cleavable amino acid sequence.

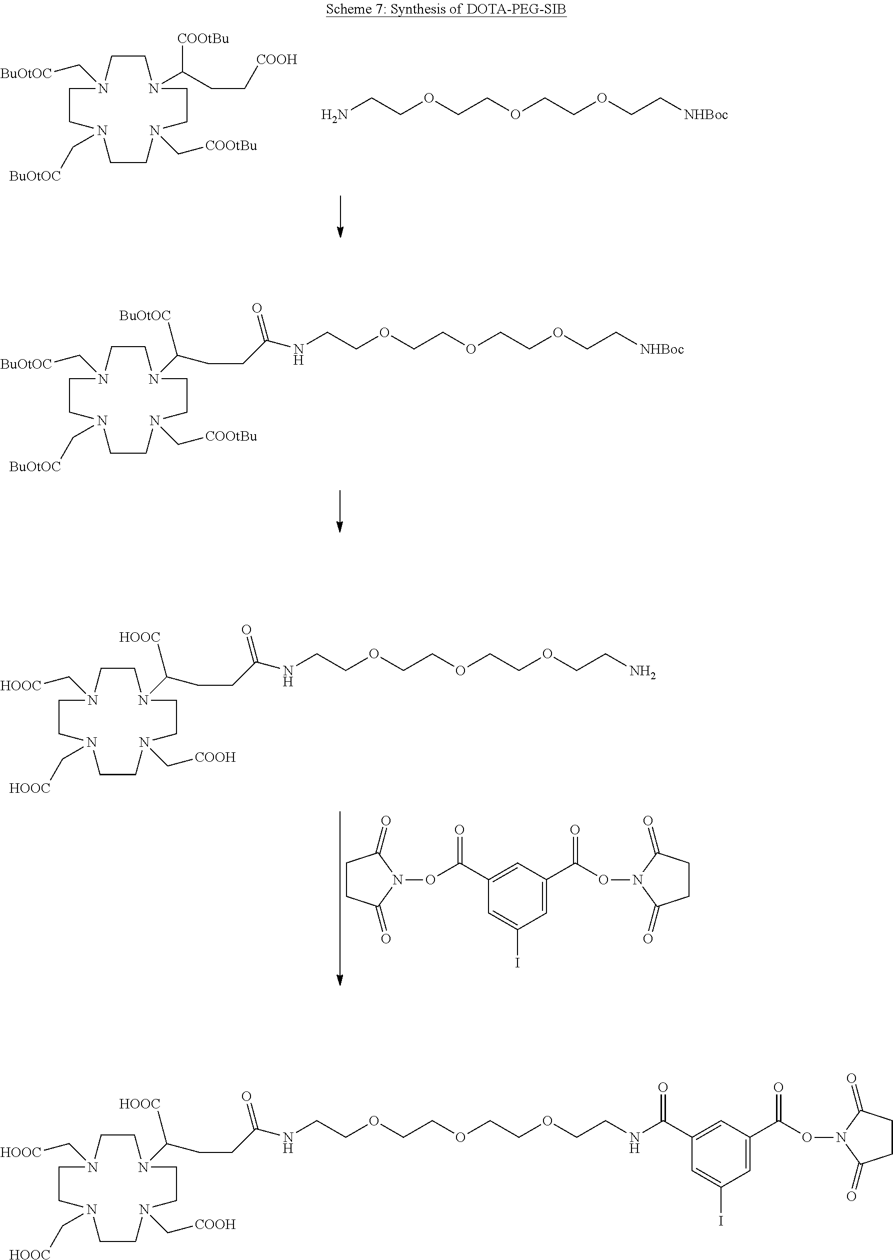

A mixture of 5-(tert-butoxy)-5-oxo-4-(4,7,10-tris(2-(tert-butoxy)-2-oxoethyl)-1,4,7,10-tetraazacyclododecan-1-yl)pentanoic acid (DOTAGA tetra-t-Bu ester; 30 mg, 43 μmol), N-hydroxysuccinimide (13.8 mg, 120 μmol), N-Boc-2-{2-[2-(2-amino-ethoxy)-ethoxy]-ethoxy}-ethylamine (35 mg, 120 μmol), and EDC (18.6 mg, 120 μmol) in DMF (0.5 mL) is stirred at 20° C. overnight. It is then purified by semi-preparative reversed-phase HPLC to obtain tri-tert-butyl 2,2′,2″-(10-(2,2,24,24-tetramethyl-4,18,22-trioxo-3,8,11,14,23-pentaoxa-5,17-diazapentacosan-21-yl)-1,4,7,10-tetraazacyclododecane-1,4,7-triyl)triacetate as an oil (16 mg, 16 μmol, 39% yield). LRMS (LCMS-ESI) m/z: 975.7 (M+H)⁺. Trifluoroacetic acid (300 μl) is added to the above product (16 mg, 16 μmol) and the resultant solution stirred at 20° C. overnight. TFA is evaporated to give 2,2′,2″-(10-(1-amino-16-carboxy-13-oxo-3,6,9-trioxa-12-azahexadecan-16-yl)-1,4,7,10-tetraazacyclododecane-1,4,7-triyl)triacetic acid as an oil (10 mg, 15.4 μmol, 96% yield). LRMS (LCMS-ESI) m/z: 651.3 (M+H) +. The above product is coupled to bis(2,5-dioxopyrrolidin-1-yl) 5-iodoisophthalate by reacting one equivalent of each reagent as well as one equivalent of N,N-diisopropylethylamine in DMF. The product is purified by reversed-phase HPLC and conjugated with a macromolecule for subsequent labeling with a radiometal such as ¹⁷⁷Lu.

### Example 4: Preconjugation-Concept

The previous examples illustrate approaches that consist of first synthesizing the radiohalogenated molecule (from a tin or other alkylmetal precursor) and then coupling the radiolabeled molecule to a macromolecule. The alternative approach is to first react the precursor for radiohalogen with the macromolecule and then radiolabel this protein-precursor conjugate. This second approach is called preconjugation and has several potential advantages including decreasing synthesis time (important with radioactivity) and increasing overall yields. In the preconjugation alternative, which is the general approach for radiometal but not radiohalogen labeling because of the difference in their chemistries, the tin-containing precursor molecule is first conjugated to the macromolecule. Then such derivatized macromolecules can be radiohalogenated, with this procedure preferably being performed at a pH lower than 6.5. This approach is illustrated in Scheme 8 using the agent shown in Scheme 7 (complexed with nonradioactive lutetium).

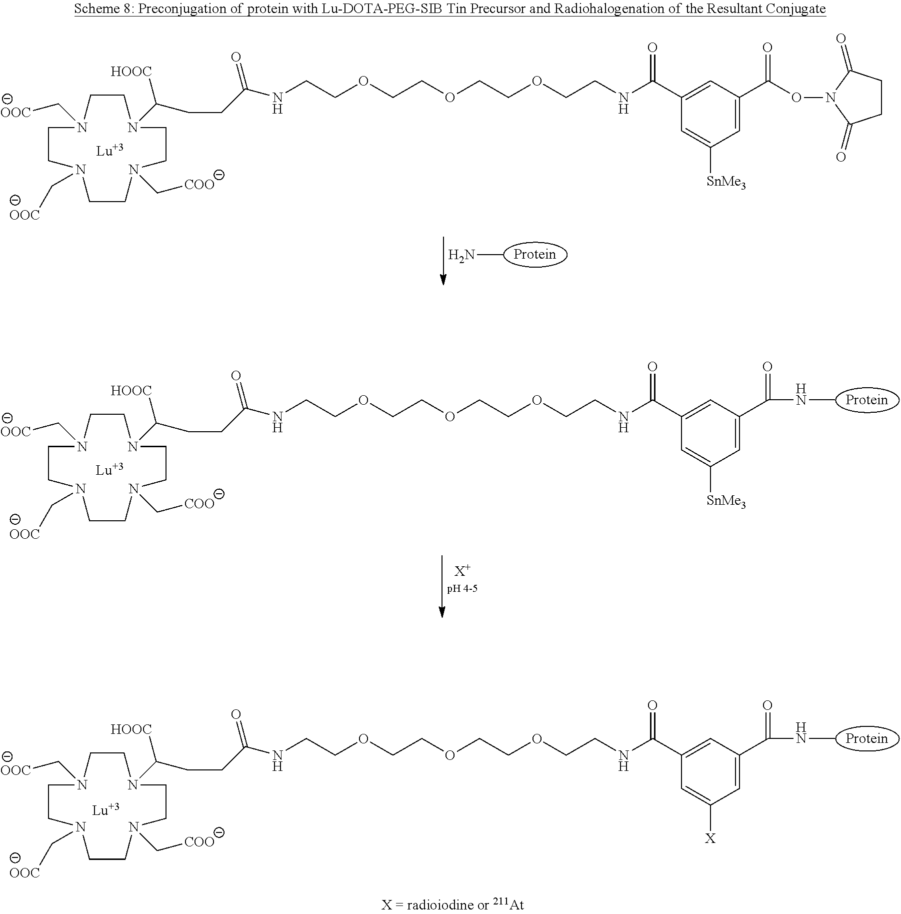

Alternatively, the iodo derivative with an uncomplexed DOTA moiety can used for labeling with radiometals such as ¹⁷⁷Lu. For example, in the case of ¹⁷⁷Lu, ¹⁷⁷LuCl₃ (2 Ci/ml, 10 μl in 0.05 M HCl is diluted with 0.15 M ammonium acetate buffer and reacted with 100-1000 μg of DOTA-PEG-SIB and when the reaction has run to completion, purified by standard size exclusion chromatography methods.

Furthermore, the tin derivative with the uncomplexed DOTA moiety can be conjugated with the macromolecule and then can be labeled with both a radiometal and a radiohalogen.

### Example 5: Preconjugation-Experimental Approach

The DOTAGA derivative 2,2′,2″-(10-(1-amino-16-carboxy-13-oxo-3,6,9-trioxa-12-azahexadecan-16-yl)-1,4,7,10-tetraazacyclododecane-1,4,7-triyl)triacetic acid is coupled to bis(2,5-dioxopyrrolidin-1-yl) 5-(trimethylstannyl)isophthalate following the same procedure described above for the iodo derivative. It is then complexed with non-radioactive lutetium. For this, 50 μmol of the tin derivative is treated with 5 equivalents of LuCl₃ in 10 ml of 0.4 M acetate buffer, pH 5.2. The progress of the reaction is followed by reversed-phase HPLC and the lutetium complex is purified by semi-preparative reversed-phase HPLC. The complex is then conjugated to a macromolecule. For this, a solution of the macromolecule in 0.2 M sodium carbonate buffer, pH 8.5 (10 nmol/ml) is added to a solution of the prosthetic agent in DMSO (25 mM; 5 μl, 125 nmol), and the mixture incubated at 20° C. for 1 h. The resultant macromolecule-prosthetic group conjugate is isolated and at the same time buffer exchanged to 0.2 M acetate, pH 5.5, by filtering through a VivaSpin ultra filtration unit with appropriate molecular weight cut off (for example, 10 kDa for VHH) (GE Healthcare). The modified macromolecule is then radiohalogenated at a pH of 5.

### Example 6: Pre-Iodination of Macromolecules With Carrier Iodine Before Radioiodination

In the strategy described above-pre-conjugating the alkyl metal prosthetic agents and subsequently performing radiohalogenation-one drawback especially for radioiodination, is that constituent tyrosine residues that are present in the macromolecule also can get radioiodinated in addition to the intended sites for radiolabeling, namely the moieties bearing the alkyl metal group. The problem with putting the radioiodine on the tyrosines is that the radioactivity would come off once in the body due to the action of endogenous deiodinases, and not be localized with the macromolecule at the cancer cells. Although this can be minimized by conducting the radioiodination at a lower pH (4-5), it cannot be completely avoided. One approach to avoid this potential problem is to introduce non-radioactive iodine onto those tyrosine residues first, before subjecting the macromolecule to radioiodination. It is highly likely that, mediated by these same endogenous deiodinases, the nonradioactive iodine on the constituent tyrosine residues would be removed, thereby restoring the original tyrosine structure and maintaining the affinity of the macromolecule for the envisioned target. Non-radioactive iodination of the proteins can be simply accomplished by treating the protein with an excess of sodium iodide in the presence of an oxidizing agent such as chloramine-T.

As an example of this approach, a VHH protein in 0.5 M sodium phosphate buffer, pH 7.4 is reacted with 15 equivalents each of sodium iodide and chloramine-T at room temperature for 5-10 min. The reaction is quenched by the addition of sodium bisulphite (2 molar equivalent of chloramine-T). The iodinated protein is purified by gel filtration or ultra-filtration.

### Example 7

Targeted Radiotherapy for CNS Disease. An attractive strategy for treating cancers in the central nervous system (CNS) is targeted radiotherapy, which uses a vector such as a small biomolecule of the invention to selectively deliver a radionuclide to malignant cell populations. An advantage of targeted radiotherapy is that one can select a radionuclide with properties that are best matched to the constraints of the intended clinical application, which for CNS tumors means selecting radiation with a tissue range that minimizes irradiation of normal CNS tissues. For example, neoplastic meningitis (NM) presents as free-floating cancer cells in the CSF and sheet-like deposits on compartmental walls. Radiation dosimetry calculations indicate that radionuclides emitting short-range radiation are best for treating NM by maximizing radiation dose deposition to tumor cells while minimizing dose to spinal cord.

VHH molecules. Also known as single-domain antibody fragments (sdAb) or nanobodies, VHH molecules are derived from Camelidae and are the smallest antigen-binding fragment of a natural antibody having a molecular weight (˜15 kDa) an order of magnitude smaller than intact mAbs. Unlike artificial Affibody scaffolds, VHHs are easily generated in nanomolar to picomolar affinity by cloning from immunized camels or llamas and selection by phage display panning. Compared with other small protein-based targeting vectors, VHHs generally offer significant advantages in terms of thermal and chemical stability, low immunogenicity, solubility, expression yields, construction of multimers as well as the ability to recognize hidden or uncommon epitopes. VHHs in both monomeric and multimeric format currently are undergoing clinical evaluation as therapeutics for a number of diseases including inflammation. A panel of anti-HER2 VHHs have been labeled with a variety of radionuclides including ^{99m}Tc, ⁶⁸Ga, ¹⁸F, ¹³¹I, and ¹⁷⁷Lu. These radiolabeled VHHs exhibited peak tumor uptake in the range of 3-6% ID/g and rapid clearance from all normal tissues except kidneys. The present invention provides more potent radiolabeled biomolecules that will exhibit significantly higher tumor uptake, lower accumulation in normal tissues including the kidneys, improved radiolabeling efficiency, and are for use in targeting internalizing receptors such as HER2 and HER1.

Alpha-Particle Emitters: Rationale for CNS Tumor Targeted Radiotherapy. Beta emitters such as ¹³¹I, like the external beam radiation used in current CNS tumor treatments, are radiation of low energy transfer. On the other hand, α-particles are high linear energy transfer (LET) radiation, with the result that their ability to kill cancer cells is not compromised by hypoxia, dose rate effects or cell cycle position, enhancing their attractiveness for targeted radiotherapy of CNS tumors. Unlike the case with low LET radiation, resistance mechanisms do not limit the effectiveness of α-particles because cells have only a limited capacity to repair DNA double-strand breaks induced by α-particles, which have also been shown to kill tumor cells by apoptotic mechanisms. The range of α-particles in tissue is only about 50-80 μm, equivalent to only a few cell diameters, which should be ideally suited for the destruction of free floating tumor cells in the CSF, thin sheets of tumor on the spinal cord, and intracranial metastases while minimizing irradiation of tumor-adjacent normal CNS tissue. Therefore, both beta and alpha emitters are encompassed by the present invention.

### Example 8: Radiolabeled iso-SAGMB and iso-SGMIB as Prosthetic Agents for Targeted Radiotherapy of HER-2 Expressing Cancers

### 1. Introduction

Human epidermal growth factor receptor 2 (HER2) is overexpressed in a subset of patients with multiple types of cancers including breast, non-small cell lung, gastric, colon and ovarian. Up to 20-30% of breast cancers overexpress HER2 and HER2 expression has been shown to confer a more aggressive phenotype, including a greater propensity to metastasize to the central nervous system (CNS). Moreover, a higher incidence of brain metastases and leptomeningial carcinomatosis have been reported in patients treated with the anti-HER2 monoclonal antibody (mAb) trastuzumab. Trastuzumab frequently prolongs survival by controlling systemic disease in many patients; however, this increases the opportunity for CNS lesions, against which trastuzumab is ineffective because of poor delivery due to the blood brain barrier impermeability of this large protein.

Patients with HER2-positive CNS disease have a grim prognosis; thus, there is a dire need for treatments that can be more effective without compromising neurologic function, which can be an unfortunate side effect of nonspecific treatments including conventional radiation therapy. An attractive approach for increasing the specificity of cancer treatment is targeted radiotherapy, in which a mAb or other vector is used to selectively deliver a cytotoxic radionuclide to cancer cells. In the context of disease within the CNS, α-particles, a radiation with a tissue range of only few cell diameters (50-80 μm), could be advantageous because it could minimize cross fire irradiation of normal tissue. Moreover, α-particles have a high relative biological effectiveness, requiring only a few traversals per cell to achieve its destruction.

As an initial investigation of the therapeutic potential of α-particles for the treatment of HER2-positive cancers, trastuzumab was labeled with the 7.2-h half-life a-emitter ²¹¹At and its cytotoxicity for 3 HER2-expressing human breast carcinoma lines was evaluated in vitro. The relative biological effectiveness of ²¹¹At-labeled trastuzumab was about 10 times higher than that of conventional external beam therapy, with significant reduction in survival achieved with only a few ²¹¹At atoms bound per cell. A subsequent study was performed in a HER2-positive breast carcinomatous meningitis model to evaluate the therapeutic efficacy of a single intrathecal injection of ²¹¹At-labeled trastuzumab. Significant prolongation in median survival with some long-term survivors was observed; however, even with direct injection into the intrathecal compartment, histopathological analyses revealed that regions of the neuroaxis had escaped treatment in some animals. Intact mAbs are not ideal for use in combination with short lived a-emitters such as ²¹¹At because their large size hinders homogeneous delivery and for intravenous applications, results in slow normal tissue clearance.

To overcome these limitations, a variety of smaller HER2-targeted proteins have been developed including recombinant fragments such as diabodies and minibodies, and smaller scaffolds such as affibodies. Another attractive platform for targeted radiotherapy, derived from Camelidae heavy-chain only antibodies and known as single domain antibody fragments (sdAbs), variable domain of heavy-chain only antibodies (VHH) or nanobodies has a molecular weight of 12-15 kDa. These VHHs can be generated relatively inexpensively with nM to pM affinity, high thermal and chemical stability, and low immunogenicity. Moreover, because of their small size, they clear rapidly from blood and normal tissues and efficiently penetrate tumors, properties that are particularly advantageous for use with short-lived α-emitters like ²¹¹At. Finally, several VHHs with high affinity for HER2 have been generated and reported to target HER2-positive cancers in animal models and in a recent clinical imaging trial.

The potential utility of the reagent, N-succinimidyl 3-[²¹¹At]astato-4-guanidinomethyl benzoate ([²¹¹At]SAGMB), as well as a novel residualizing agent, N-succinimidyl 3-[²¹¹At]astato-5-guanidinomethyl benzoate (iso-[²¹¹At]SAGMB), for labeling 5F7 VHH with ²¹¹At was evaluated. In parallel, the potential utility of the analogous reagents —N-succinimidyl 4-guanidinomethyl-3-[¹³¹I]iodobenzoate ([¹³¹I]SGMIB) and N-succinimidyl 3-guanidinomethyl-5-[¹³¹I]iodobenzoate (iso-[¹³¹I]SGMIB)-labeled with the beta-particle emitter ¹³¹I were evaluated. Tumor targeting properties of the four residualizing agents were evaluated in HER2-expressing breast carcinoma cells and xenografts.

### 2. Materials and Methods

### 2.1. General

All reagents were purchased from Sigma-Aldrich except where noted. Sodium [¹³¹I]iodide (44.4 TBq/mmol) in 0.1 N NaOH was obtained from Perkin-Elmer Life and Analytical Sciences (Boston, MA, USA). Astatine-211 was produced on the Duke University CS-30 cyclotron via the ²⁰⁹Bi(α, 2n)²¹¹At reaction by bombarding natural bismuth metal targets with 28 MeV α-particles. Astatine-211 was isolated from the target by dry distillation, trapped in PEEK or PTFE tubing and finally extracted with a solution of N-chlorosuccinimide (NCS) in methanol (0.2 mg/mL) as described previously. Succinimidyl 4/5-((1, 2-bis(tert-butoxycarbonyl)guanidino)methyl)-3-iodobenzoate (Boc₂-SGMIB/iso-SGMIB) and their corresponding tin precursors (Boc₂-SGMTB/iso-SGMTB) were synthesized as reported before. High-performance liquid chromatography (HPLC) was performed using a Beckman Gold HPLC system equipped with a Model 126 programmable solvent module, a Model 166 NM variable wavelength detector, and a ScanRam RadioTLC scanner/HPLC detector combination (LabLogic; Brandon, FL, USA). HPLC data were acquired and processed using the Laura software (LabLogic). Normal-phase HPLC was performed using a 4.6×250 mm Partisil silica column (10 μm: Alltech, Deerfield, IL, USA), eluted in isocratic mode with a mixture of 0.2% acetic acid in 75:25 hexanes: ethyl acetate (v/v) at a flow rate of 1 mL/min. Disposable PD 10 desalting columns for gel filtration were purchased from GE Healthcare (Piscataway, NJ, USA). Instant thin layer chromatography (ITLC) was performed using silica gel impregnated glass fiber sheets (Pall Corporation, East Hills, NY, USA) with PBS, pH 7.4 as the mobile phase. Developed sheets were analyzed for radioactivity either using the TLC scanner described above or by cutting the sheet into small strips and counting them in an automated gamma counter. Radioactivity levels in various samples were assessed using either an LKB 1282 (Wallac, Finland) or a Perkin Elmer Wizard II (Shelton, CT, USA) automated gamma counter.

### 2.2. Anti-HER2 5F7 VHH Molecule

The anti-HER2 5F7 VHH molecule was obtained as a gift from Ablynx NV (Ghent, Belgium), was selected from phage libraries derived from llamas that had been immunized with SKBR3 human breast carcinoma cells. Its production, purification and characterization were as described previously (see Pruszynski M, Koumarianou E, Vaidyanathan G, Revets H, Devoogdt N, Lahoutte T, et al. Targeting breast carcinoma with radioiodinated anti-HER2 Nanobody. Nucl Med Biol 2013; 40:52-9, which is incorporated herein by reference), except that the glycine-glycine-cysteine (GGC) C-terminus tail was omitted, resulting in a purely monomeric preparation.

### 2.3. Cells and Cell Culture Conditions

Cell culture reagents were purchased from Invitrogen (Grand Island, NY, USA). BT474M1 human breast carcinoma cells were grown in DMEM/F12 medium containing 10% fetal calf serum (FCS), streptomycin (100 μg/mL), and penicillin (100 IU/mL) (Sigma-Aldrich, MO, USA). Cells were cultured at 37° C. in a 5% CO₂ humidified incubator.

2.4. Synthesis of [¹³¹I]SGMIB and iso-[¹³¹I]SGMIB

In most experiments, [¹³¹I] SGMIB and iso-[¹³¹I]SGMIB were synthesized as reported previously by the radioiododestannylation of the corresponding tin precursor using tert-butyl hydroperoxide (TBHP) as the oxidant and chloroform as the solvent. See Vaidyanathan G, Zalutsky M R. Synthesis of N-succinimidyl 4-guanidinomethyl-3-[*I]iodobenzoate: a radio-iodination agent for labeling internalizing proteins and peptides. Nature Prot 2007; 2:282-6 and Choi J, Vaidyanathan G, Koumarianou E, McDougald D. Pruszynski M, Osada T, et al. N-Succinimidyl guanidinomethyl iodobenzoate protein radiohalogenation agents: influence of isomeric substitution on radiolabeling and target cell residualization. Nucl Med Biol 2014; 41:802-12, which are incorporated herein by reference. In more recent runs, NCS was used as the oxidant and the reaction was performed in methanol. For this, a solution of NCS in methanol (0.2 mg/mL; 100 μL), acetic acid (1 μL) and [¹³¹I]iodide (1-2 μL; 37-74 MBq) were added in that order to a half-dram glass vial containing 50 μg of the required tin precursor, and the reaction was allowed to proceed at 20° C. for 15 min with occasional swirling of the vial. Most of the solvent was evaporated with a stream of argon, and the residue partitioned between 200 L each of ethyl acetate and water. The ethyl acetate layer was separated, dried with anhydrous sodium sulfate and the ethyl acetate was evaporated. The residual radioactivity was reconstituted in the HPLC mobile phase (200 μL) and injected onto a normal phase column. Procedures for isolation and deprotection were as described below for [²¹¹At]SAGMB and iso-[²¹¹At]SAGMB.

2.5. Synthesis of [²¹¹At]SAGMB and iso-[²¹¹At]SAGMB

Astatine-211 in NCS/methanol (30-56 MBq) was added to a vial containing 200 μg of the required tin precursor followed by 10 μL acetic acid. The reaction mixture was incubated at 20° C. for 30 min and methanol was evaporated with a gentle stream of argon. The residual mixture was re-dissolved in 20 μL of (75:25) hexanes/ethyl acetate and injected onto the normal phase HPLC column. The HPLC fractions containing Boc₂-iso-[²¹¹At]SAGMB or Boc₂-[²¹¹At]SAGMB (t_{R}=˜25 min) were isolated, and the solvents from these were evaporated under a stream of argon for 20 min. Boc protecting groups were removed by treatment with 100 μL of trifluoroacetic acid (TFA) at 20° C. for 10 min. To insure complete removal of TFA, the process of ethyl acetate addition (50 μL) and evaporation was performed three times. The residual radioactivity was then used as such for 5F7 VHH labeling.

### 2.6. Radiolabeling of 5F7 VHH

lodine-131 labeling of 5F7 VHH with [¹³¹I]SGMIB or iso-[¹³¹I]SGMIB was performed as reported previously. See Choi J, Vaidyanathan G, Koumarianou E, McDougald D, Pruszynski M, Osada T, et al. N-Succinimidyl guanidinomethyl iodobenzoate protein radiohalogenation agents: influence of isomeric substitution on radiolabeling and target cell residualization. Nucl Med Biol 2014; 41:802-12, which is incorporated herein by reference. For ²¹¹At-labeling, a solution of 5F7 VHH in 0.1 M borate buffer, pH 8.5 (50 μL, 2 mg/mL) was added to the vial containing the [²¹¹At]SAGMB or iso-[²¹¹At]SAGMB activity and the mixture was incubated at 20° C. for 20 min. The labeled 5F7 VHH was purified by gel filtration over a PD-10 column eluted with phosphate buffered saline (PBS). Before use, the PD-10 column was preconditioned with human serum albumin to minimize nonspecific binding.

### 2.7. Quality Control Procedures

Each ¹³¹I- and ²¹¹At-labeled 5F7 preparation was evaluated by ITLC and SDS-PAGE to determine protein associated radioactivity, and the presence of aggregates and multimeric species, respectively. For ITLC, PBS, pH 7.4, was used as the mobile phase; with this system, intact protein remained at the origin (R_{f}=0) and lower molecular weight radioactive species moved with an R_{f} value of 0.7-0.8. SDS-PAGE under non-reducing conditions and phosphor imaging were performed as previously described. The immunoreactive fractions of the labeled 5F7 VHH conjugates were determined by the Lindmo method using magnetic beads coated with HER2 extracellular domain, or as a negative control, bovine serum albumin (BSA). Briefly, aliquots of labeled 5F7 (˜5 ng) were incubated with doubling concentrations of both HER2- and BSA-coated beads, and the immunoreactive fraction was calculated as the specific binding extrapolated to infinite HER2 excess.

### 2.8. Binding Affinity of Radiolabeled 5F7 Conjugates

BT474M1 breast carcinoma cells were plated in 24-well plates at a density of 8×10⁴ cells/well and incubated at 37° C. for 24 h. The cells were then allowed to acclimatize at 4° C. for 30 min prior to the addition of increasing concentrations of radiolabeled 5F7 conjugates (0.1-100 nM). Cells were then incubated at 4° C. for 2 h, the medium containing unbound radioactivity was removed, and the cells were washed twice with cold PBS. Finally, the cells were solubilized by treatment with IN NaOH (0.5 mL) at 37° C. for 10 min. Cell-associated radioactivity was counted using an automated gamma counter. To determine non-specific binding, a parallel assay was performed as above except that a 100-fold excess of trastuzumab also was added to the incubation medium. The data were fit using GraphPad Prism software to determine the K_{d} values.

### 2.9. Internalization Assays

Internalization and cell processing assays were performed in paired-label format using BT474M1 breast carcinoma cells. Cells at density of 8×10⁵ cells per well in 3 mL medium were plated in 6-well plates and after overnight incubation at 37° C., were brought to 4° C. and incubated for 30 min. Medium was removed and replenished with fresh medium containing 5 nmol each of either [²¹¹At]SAGMB-5F7 plus [¹³¹I] SGMIB-5F7, or iso-[²¹¹At]SAGMB-5F7 plus iso-[¹³¹I]SGMIB-5F7, and the cells were further incubated at 4° C. for 1 h. Cell culture supernatants containing unbound radioactivity were removed and fresh medium at 37° C. was added. The fraction of initial cell-bound radioactivity that was internalized, on the cell membrane, or released into the cell culture supernatant after incubation at 37° C. for 1, 2, 4, 6, and 24 h was determined as described previously. To determine nonspecific uptake, parallel experiments were performed as above except that a 100-fold molar excess of trastuzumab also was added to the wells.

### 2.10. Paired-Label Biodistribution Experiments

Animal experiments were performed following the guidelines established by the Duke University Institutional Animal Care and Use Committee. Subcutaneous BT474M1 tumor xenografts were established in SCID mice as described previously (see Pruszynski M, Koumarianou E, Vaidyanathan G, Revets H, Devoogdt N, Lahoutte T, et al. Improved tumor targeting of anti-HER2 nanobody through N-succinimidyl 4-guanidinomethyl-3-iodobenzoate radiolabeling. J Nucl Med 2014; 55:650-6, which is incorporated herein by reference) and two paired-label biodistribution studies were performed when tumors reached a volume of about 350-500 mm³. Groups of 5 mice received tail vein injections of ˜185 kBq each of the labeled molecules. In the first experiment, [²¹¹At]SAGMB-5F7 (178 MBq/mg) and [¹³¹I]SGMIB-5F7 (174 MBq/mg) were administered, and in the second, iso-[²¹¹At]SAGMB-5F7 (85 MBq/mg) and iso-[¹³¹I]SGMIB-5F7 (89 MBq/mg) were injected. In this way, the effect of ²¹¹At-for-¹³¹I substitution on tumor targeting and in vivo stability for each of the two isomer configurations could be directly compared. Biodistribution was evaluated at 1 h, 2 h, 4 h, and 21 h after injection; an additional time point of 14 h was included in the second study. Blood and urine were collected, and mice were killed by an overdose of isofluorane. Tumor and normal tissues were isolated, blot-dried, and weighed along with blood and urine. All tissue samples together with 5% injection standards were counted for ¹³¹I and ²¹¹At activity using an automated gamma counter, and the percentage of injected dose (% ID) per organ and per gram of tissue were calculated.

### 2.11. Statistical Analyses

Data are presented as mean ±standard deviation. Differences in the behavior of co-incubated (in vitro) or co-administered (in vivo) labeled conjugates were analyzed for statistical significance with a paired two-tailed Student t-test using the Microsoft Office excel program, while differences in the behavior of labeled conjugates that were not co-incubated or co-administered were tested with an unpaired Student t-test. Differences with a P value<0.05 were considered statistically significant.

### 3. Results

### 3.1. Radiolabeling

The scheme for synthesis of the four radiohalogenated 5F7 VHH conjugates is provided in Scheme 9.

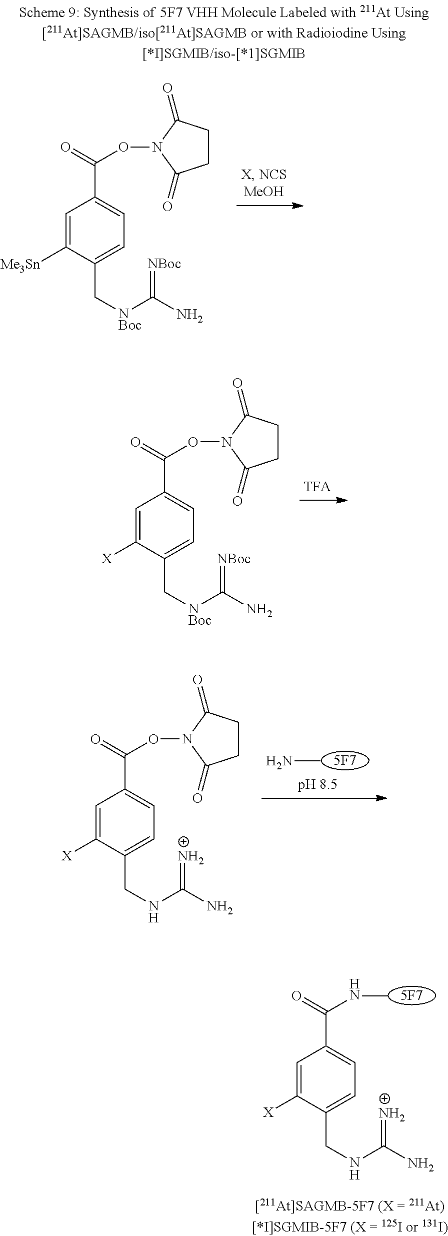

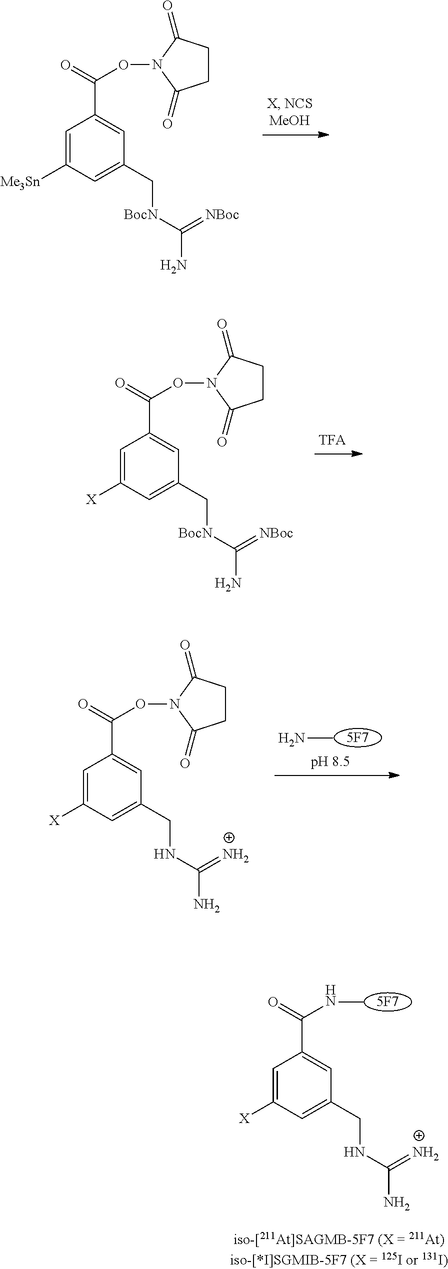

The radiochemical yield for the synthesis of iso-[²¹¹At]SAGMB-Boc₂ was 66.8±2.4% (n=7) compared with 62.6±2.3% (n=6) for [²¹¹At]SAGMB-Boc₂ under identical conditions. Although the difference in the two yields was small, it was statistically significant (P<0.05). The radiochemical yield for the synthesis of [²¹¹At]SAGMB-Boc₂ was similar to that reported previously when TBHP was used as the oxidant and chloroform as the solvent. In most experiments reported herein, [¹³¹I]SGMIB and iso-[¹³¹I]SGMIB were synthesized using TBHP as the oxidant; however, in a few studies, [¹³¹I]SGMIB and iso-[¹³¹I]SGMIB were synthesized using NCS as the oxidant and methanol as the solvent, which resulted in radiochemical yields of 69.2±4.2% (n=4) and 84.0±4.5% (n=2), respectively, considerably higher than those obtained using TBHP and chloroform.

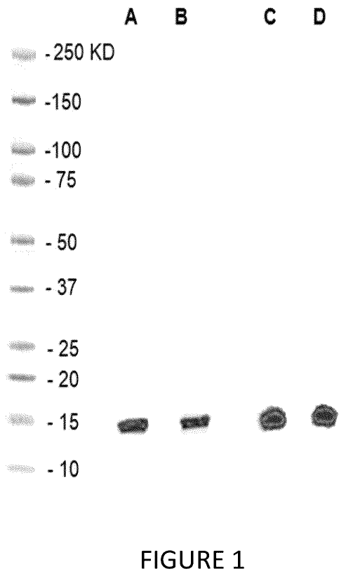

Labeling 5F7 VHH with ²¹¹At was accomplished by reaction with [²¹¹At]SAGMB and iso-[²¹¹At]SAGMB, which were obtained by treatment of Boc₂-[²¹¹At]SAGMB and Boc₂-iso-[²¹¹At]SAGMB with TFA. When performed under identical conditions, the conjugation efficiency of iso-[²¹¹At]SAGMB (39.5±6.8%; n=5) and [²¹¹At]SAGMB (38.4±15.6%; n=6) to 5F7 was similar (P>0.05). Conjugation efficiencies for labeling 5F7 with [¹³¹I]SGMIB and iso-[¹³¹I]SGMIB were 28.9±13.0% (n=6) and 33.1±7.1% (n=6), respectively. The radiochemical purity obtained by ITLC analysis was 98.9%, 97.8%, 98.6%, and 98.4% for iso-[²¹¹At]SAGMB-5F7, [²¹¹At]SAGMB-5F7, iso-[¹³¹I]SGMIB-5F7 and [¹³¹I]SGMIB-5F7, respectively. As shown in FIG. 1, SDS-PAGE performed under non-reducing conditions demonstrated that more than 98% of the radioactivity for the 4 radiohalogenated 5F7 conjugates was present in a single band with a molecular weight of about 15 kDa, corresponding to the molecular weight of a VHH monomer.

### 3.2. Immunoreactive Fraction and Binding Affinity

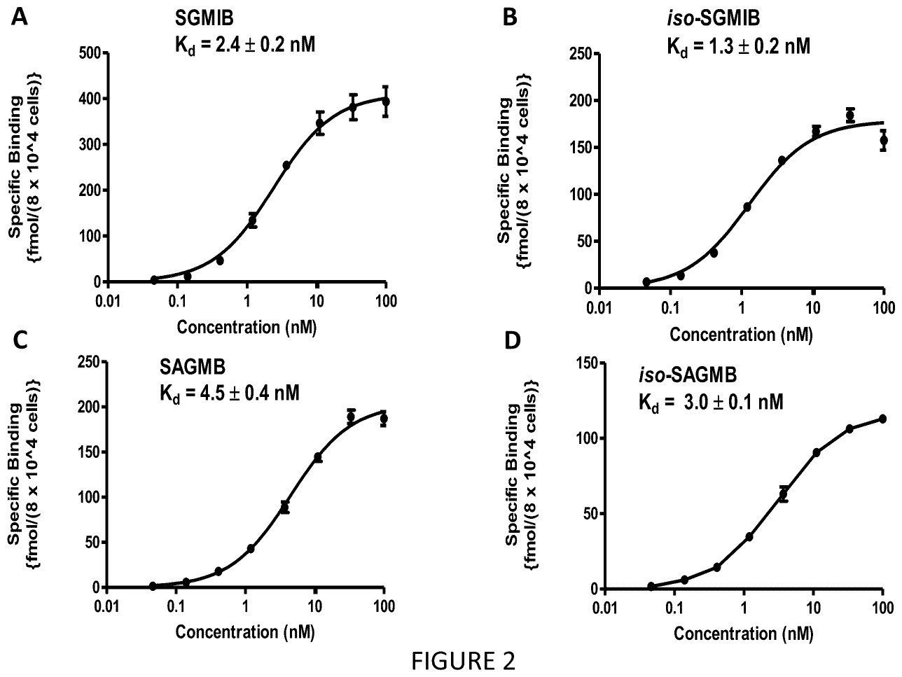

To determine whether labeling 5F7 VHH compromised HER2 binding, immunoreactive fractions were determined in paired-label format using the extracellular domain of HER2 as the molecular target. The immunoreactive fractions were determined to be 81.3±0.9%, 83.5±1.1%, 81.8±1.4% and 84.5±0.8% for iso-[²¹¹At]SAGMB-5F7, [²¹¹At]SAGMB-5F7, iso-[¹³¹I]SGMIB-5F7 and [¹³¹I]SGMIB-5F7, respectively, suggesting that 5F7 VHH retained immunoreactivity to a similar degree irrespective of the prosthetic agent used. The dissociation constant (K_{d}) values obtained from saturation binding assays performed on HER2-expressing BT474M1 human breast carcinoma cells were <5 nM for the four labeled conjugates (FIG. 2). The data of FIG. 2 was provided based on incubating cells (8×10⁴) with increasing concentrations of the labeled VHH conjugates and specific cell-associated radioactivity determined as described herein. Plots were generated and Kd values calculated using GraphPad Prism software. However, significantly higher affinity binding (P<0.05) was observed for iso-[²¹¹At]SAGMB-5F7, (3.0±0.1 nM) compared with [²¹¹At]SAGMB-5F7, (4.5±0.4 nM). The K_{d} values for iso-[¹³¹I]SGMIB-5F7 and [¹³¹I]SGMIB-5F7 were 1.3±0.2 nM and 2.4±0.2 nM, respectively, again indicating higher affinity binding for the iso-configuration conjugate. The ¹³¹I-labeled conjugates had significantly higher binding affinity than their corresponding ²¹¹At-labeled 5F7 counterparts (P<0.05).

### 3.3. Internalization Assays

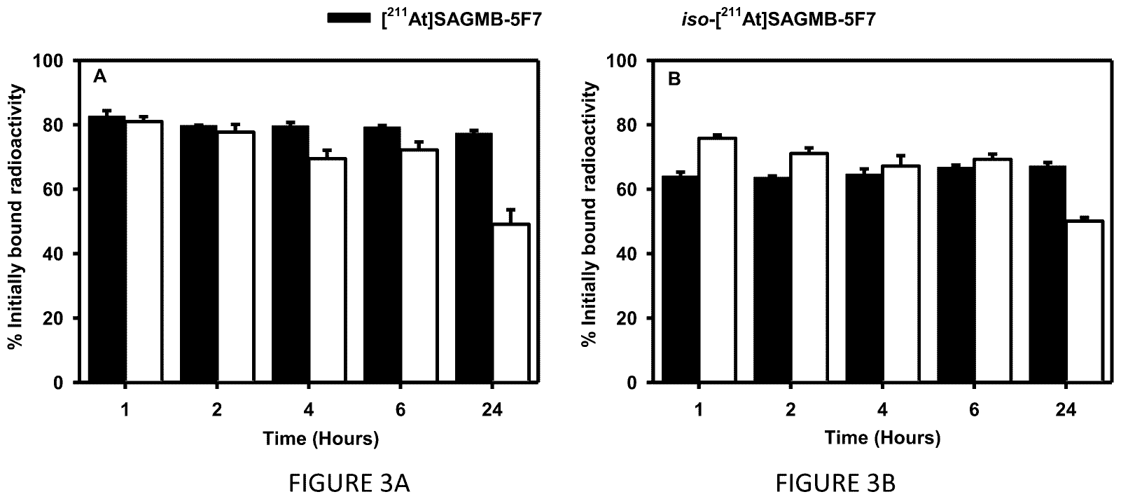

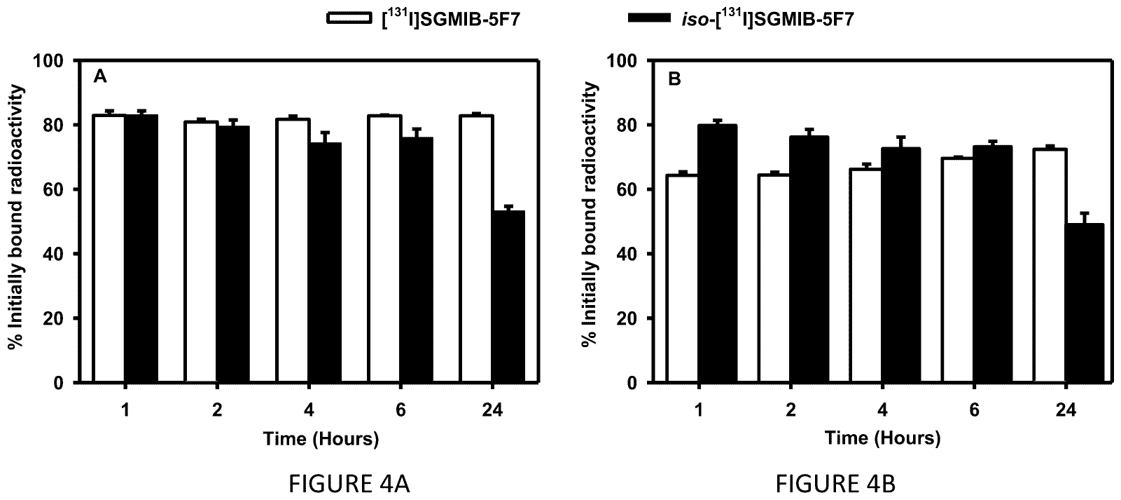

Paired-label internalization assays were performed using HER2-expressing BT474M1 cells to determine the extent of intracellular trapping of radioactivity in vitro with [²¹¹At]SAGMB-5F7 and iso-[²¹¹At]SAGMB-5F7 (FIGS. 3), and [¹³¹I]SGMIB-5F7 and iso-[¹³¹I]SGMIB-5F7 (FIG. 4). The data represented in FIG. 3 was generated based on two versions of the labeled 5F7, obtained from two different experiments. As shown in FIG. 3, the percentage of initially bound radioactivity that was cell associated (membrane bound+internalized) and internalized for [²¹¹At]SAGMB-5F7 remained nearly constant for 24 h, when values of 77.4±0.8% and 67.2±1.1%, respectively, were observed. In general, changing the nature of the prosthetic agent did not affect residualization of radioactivity in HER2-positive cancer cells. For example, at 6 h, 69.5±1.2% and 73.2±1.7% of initially bound radioactivity remained in the intracellular compartment for iso-[²¹¹At]SAGMB-5F7 and iso-[¹³¹I]SGMIB-5F7, respectively. However, unlike the behavior of [¹³¹I]SGMIB-5F7 and [²¹¹At]SAGMB-5F7, intracellular radioactivity levels from iso-[¹³¹I]SGMIB-5F7 (49.0±3.6%) and iso-[²¹¹At]SAGMB-5F7 (48.4±5.5%) at 24 h was significantly lower (P<0.05) than those observed from 1-6 h.

### 3.4. Biodistribution Studies

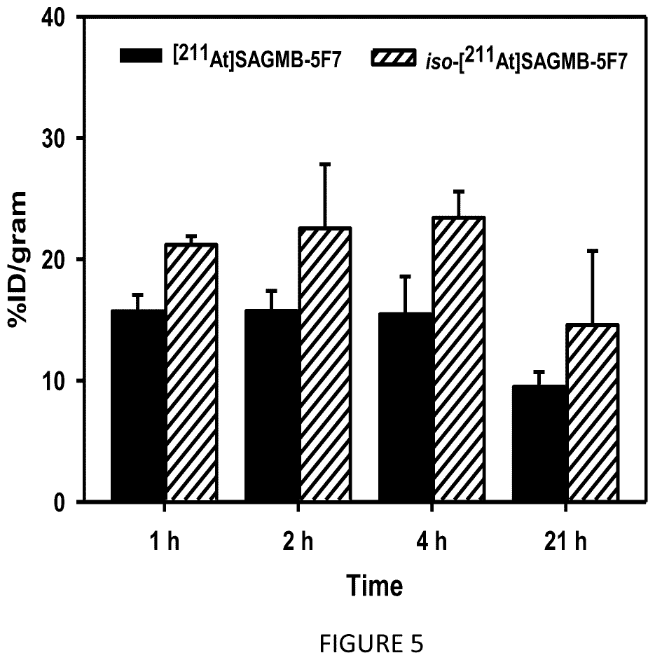

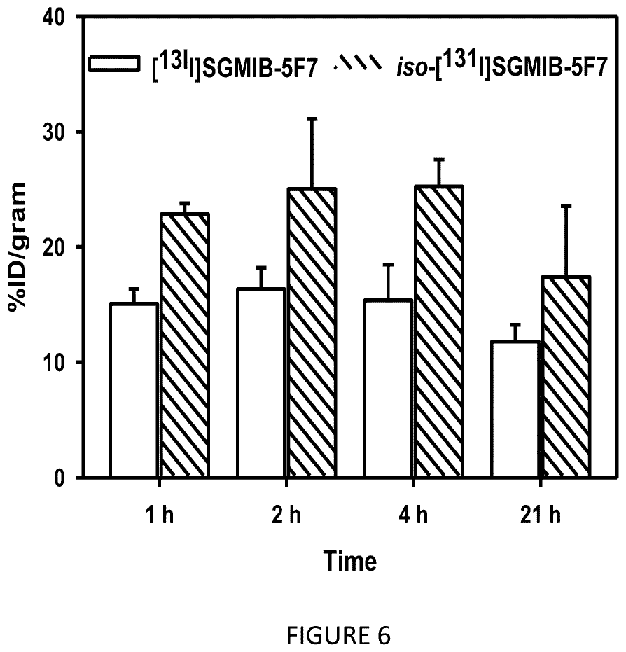

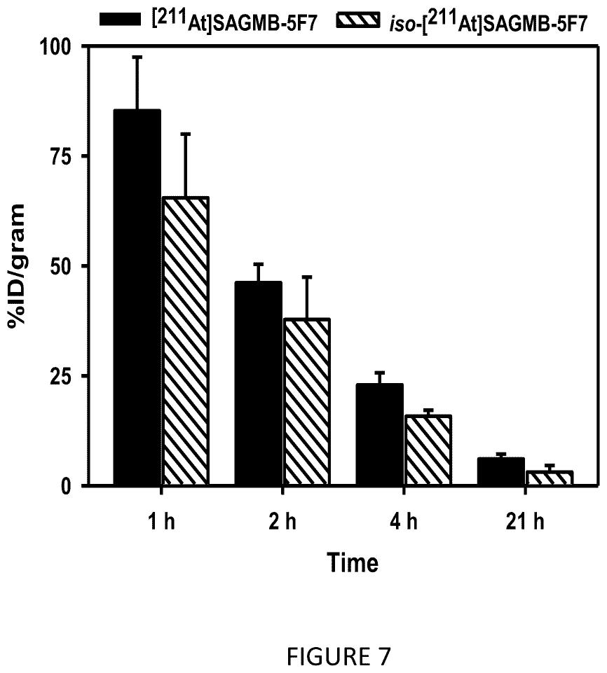

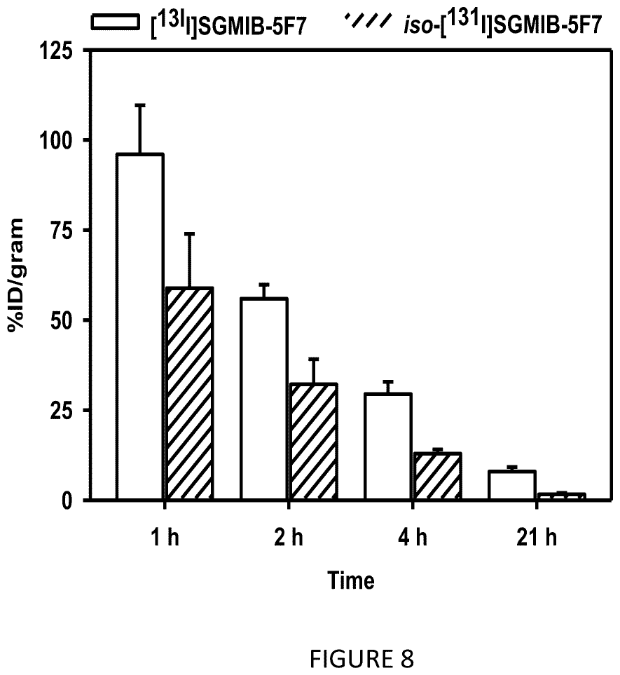

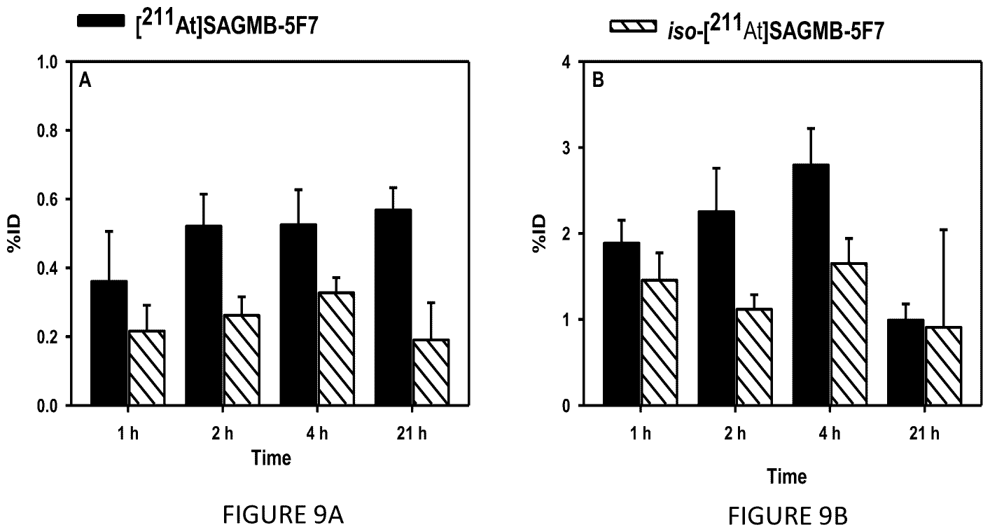

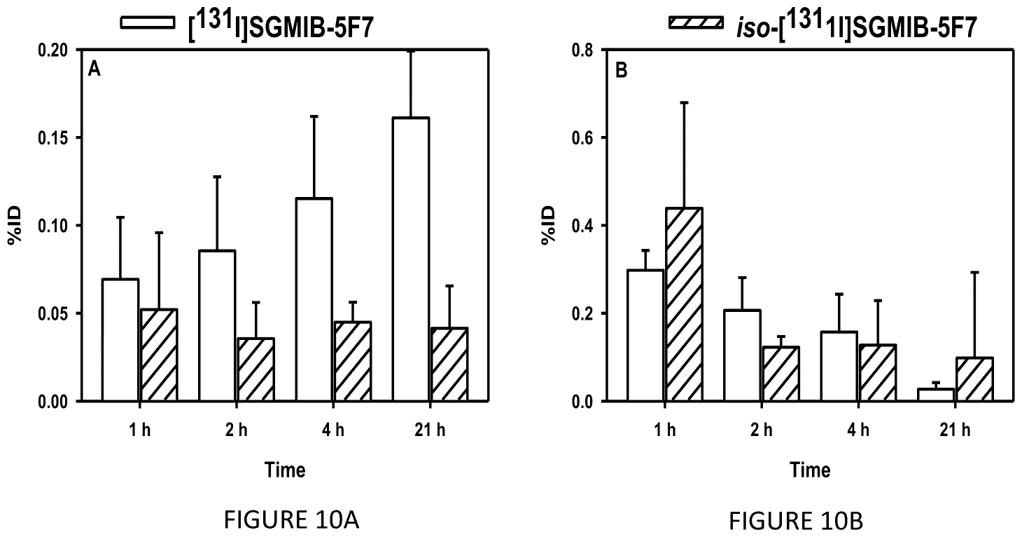

Two-paired label experiments were performed in SCID mice with subcutaneous BT474M1 breast carcinoma xenografts to directly compare the tissue distribution of [²¹¹At]SAGMB-5F7 and iso-[²¹¹At]SAGMB-5F7 to their ¹³¹I-labeled counterparts. The results obtained over a 21 h period. corresponding to approximately three half-lives of ²¹¹At decay, are summarized in Table 1 and Table 2, respectively. Tumor uptake of [²¹¹At]SAGMB-5F7 remained at 15-16% ID/g from 1-4 h post injection and then declined to 9.49±1.22% ID/g at 21 h (FIG. 5). Similar tumor uptake values were observed for co-administered [¹³¹I]SGMIB-5F7 except at 21 h (FIG. 6) when values for the radioiodinated conjugate were about 20% higher (11.8±1.5% ID/g; P<0.05). In the second experiment, similar trends were observed with regard to tumor uptake of iso-[²¹¹At]SAGMB-5F7 in comparison to its radioiodinated counterpart. However, tumor accumulation of iso-[²¹¹At]SAGMB-5F7 was almost 50% higher than that of [²¹¹At]SAGMB-5F7 at all time points (FIG. 5), peaking at 23.4±2.2% ID/g at 4 h (difference significant, P<0.05, except at 21 h by unpaired t test). Likewise, tumor uptake of iso-[¹³¹I]SGMIB-5F7 was significantly higher than that of [¹³¹I]SGMIB-5F7 at all time points (FIG. 6). With the exception of the kidneys, normal tissue uptake of the four 5F7 radioconjugates was low, particularly for iso-[²¹¹At]SAGMB-5F7 and iso-[¹³¹I]SGMIB-5F7. In kidneys, activity levels for the iso-conjugates were significantly lower than those for the corresponding non-iso-conjugate (P<0.05 by unpaired t test) (FIGS. 7 and 8), with the difference less pronounced for the ²¹¹At-labeled conjugates. Because of the lower carbon-halogen bond strength expected for ²¹¹At-labeled compounds, comparison of activity levels in the thyroid and the stomach, tissues known to sequester free astatide and iodide, can shed light on the relative in vivo stability of these conjugates. The uptake of ²¹¹At and ¹³¹I activity in thyroid and stomach after injection of the four 5F7 VHH conjugates is summarized in FIGS. 9 and 10, respectively. Thyroid and stomach accumulation for both ²¹¹At-labeled 5F7 conjugates was significantly higher than seen with their ¹³¹I-labeled co-administered counterparts. However, thyroid and stomach activity levels were about twofold lower for iso-[²¹¹At]SAGMB-5F7 compared with [²¹¹At]SAGMB-5F7, suggesting a lower degree of deastatination in vivo for iso-[²¹¹At]SAGMB-5F7.

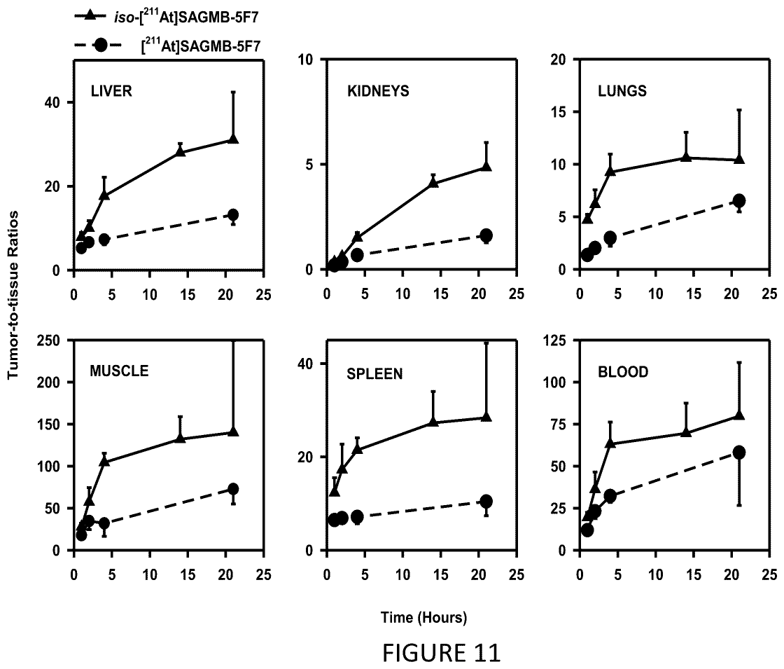

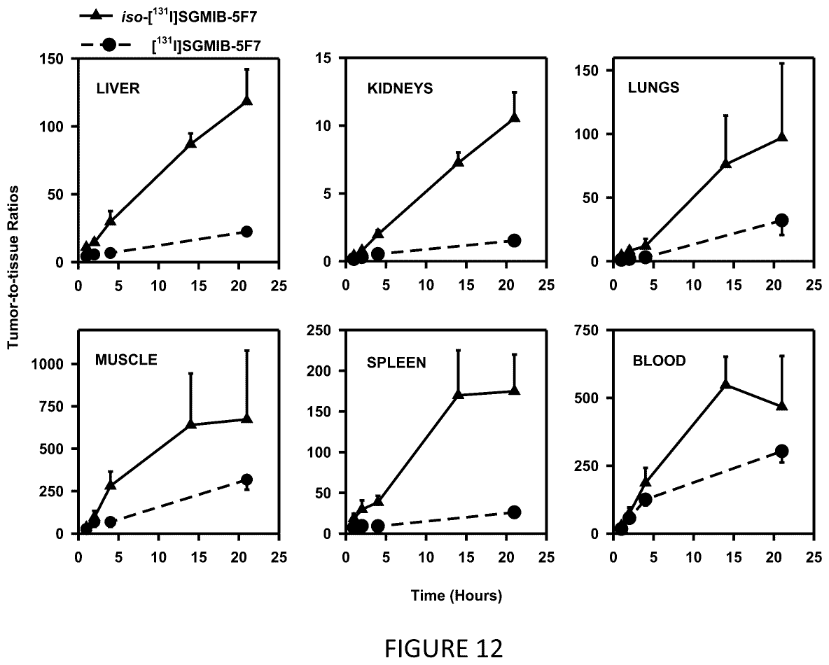

As shown in FIG. 11, tumor-to-normal tissue ratios for iso-[²¹¹At]SAGMB-5F7 were significantly higher than those for [²¹¹At]SAGMB-5F7 in all tissues. For example, tumor-to-liver, tumor-to-blood, tumor-to-spleen and tumor-to-kidney ratios were 18±4, 63±13, 21±3, and 1.50±0.25, respectively, for iso-[²¹¹At]SAGMB-5F7 at 4 h, compared with 7.31±1.26, 32±4, 7.11±1.47, and 0.67±0.08 for [²¹¹At]SAGMB-5F7. Likewise, tumor-to-normal tissue ratios for iso-[¹³¹I]SGMIB-5F7 were significantly higher than those for [¹³¹I]SGMIB-5F7 in all tissues (FIG. 12). Finally, tumor-to-normal tissue ratios for the radioiodinated 5F7 VHH conjugates were considerably higher than those for the corresponding ²¹¹At-labeled 5F7 VHH conjugates.

### 4. Discussion

In the present study, the anti-HER2 5F7 VHH was successfully labeled with the α-particle emitting radiohalogen ²¹¹At using two related prosthetic agents, [²¹¹At]SAGMB and iso-[²¹¹At]SAGMB, designed to trap the radionuclide in HER2-expressing cancer cells after receptor-mediated internalization through the generation of positively charged, labeled catabolites. The high cytotoxicity of ²¹¹At α-particles for HER2 expressing breast carcinoma cells has been demonstrated with ²¹¹At-labeled trastuzumab both in vitro and in vivo in compartmental settings. Although ²¹¹At has many potential advantages for targeted radiotherapy, the combination of the short tissue range of its α-particles and its 7.2-h half-life necessitates the development of strategies for rapidly achieving homogeneous and prolonged delivery to cancer cells with rapid clearance from normal tissues. Most approaches for achieving this goal utilize a small molecule such as a mAb fragment; however, unlike the case with whole mAbs, ²¹¹At-labeled mAb fragments exhibit high uptake in thyroid and stomach, indicating release of free ²¹¹At in vivo. Within the HER2 targeting space, this behavior has been observed with an affibody (7 kDa) labeled using N-succinimidyl 3-[²¹¹At]astatobenzoate (SAB), which exhibited 25-55 times higher stomach and thyroid levels than the corresponding ¹²⁵I-labeled construct. An anti-HER2 diabody also has been labeled with ²¹¹At using N-succinimidyl N-(4-[²¹¹At]astatophenethyl succinamate (SAPS) and although some encouraging therapeutic responses were obtained, biodistribution results for the ²¹¹At-labeled diabody were not reported.

In attempting to develop optimal ²¹¹At-labeled anti-HER2 constructs, it is important to not only consider the in vivo stability issue noted above but also how to maximize the extent and duration of radioactivity entrapment in cancer cells after binding and internalization of the labeled molecule. In addition, one must select a protein format that offers rapid tumor targeting at therapeutically relevant levels without prolonged residence times in normal tissues. The excellent results obtained with anti-HER2 VHH SGMIB conjugates provided motivation for the current study evaluating the potential utility of guanidinomethyl substituted prosthetic groups for labeling 5F7 VHH with ²¹¹At. The 5F7 VHH with (see Pruszynski M. Koumarianou E. Vaidyanathan G. Revets H. Devoogdt N. Lahoutte T. et al. Improved tumor targeting of anti-HER2 nanobody through N-succinimidyl 4-guanidinomethyl-3-iodobenzoate radiolabeling. J Nucl Med 2014;55:650-6, which is incorporated herein by reference) and without (see Vaidyanathan G. McDougald D. Choi J., Koumarianou E. Weitzel D. Osada T. et al. Preclinical evaluation of ¹⁸F-labeled anti-HER2 nanobody conjugates for imaging HER2 receptor expression by immuno-PET. J Nucl Med 2016; 57:967-73), a GGC tail has been evaluated after SGMIB labeling in SCID mice with BT474M1 xenografts and with both constructs, tumor uptake peaked 2 h after injection, suggesting that this VHH had localization kinetics compatible with the 7.2-h half-life of ²¹¹At. Because the version without the GGC tail exists as a pure monomer vs. a mixture of monomer and dimer with 5F7-GGC (see Pruszynski M. Koumarianou E.

Vaidyanathan G. Revets H. Devoogdt N. Lahoutte T. et al. Improved tumor targeting of anti-HER2 nanobody through N-succinimidyl 4-guanidinomethyl-3-iodobenzoate radiolabeling. J Nucl Med 2014;55:650-6, which is incorporated herein by reference) and exhibited significantly higher tumor localization, the tailless 5F7 construct was selected for use in these experiments.

Because of the larger size of the astatine atom compared with the iodine atom, steric hindrance could be an even more important factor for ²¹¹At labeling. Based on the significantly higher radioiodination and protein conjugation yields observed for iso-[¹³¹I]SGMIB compared with [¹³¹I]SGMIB, both iso-[²¹¹At]SAGMB (1,3,5-isomer) and [²¹¹At]SAGMB (1,3,4-isomer) were evaluated for labeling 5F7 VHH. Although radiolabeling and VHH conjugation yields for iso-[²¹¹At]SAGMB were higher than those for [²¹¹At]SAGMB. these differences were not significant. Conjugation of these prosthetic groups, as well as their radioiodinated counterparts, resulted in monomeric products with excellent immunoreactivity and affinity (<5 nM) for binding to HER2-overexpressing BT474M1 breast carcinoma cells. The results obtained for [¹³¹I]SGMIB-5F7 were in good agreement with those reported previously for the [¹³¹I]SGMIB-5F7-GGC construct. See Pruszynski M. Koumarianou E, Vaidyanathan G. Revets H. Devoogdt N. Lahoutte T. et al. Improved tumor targeting of anti-HER2 nanobody through N-succinimidyl 4-guanidinomethyl-3-iodobenzoate radiolabeling. J Nucl Med 2014;55:650-6, which is incorporated herein by reference. With both isomers, the affinity for the ²¹¹At-labeled 5F7 conjugate was about half that of the corresponding ¹³¹I-labeled 5F7 VHH conjugate. While not bound by any mechanism of action it is believed that the larger size of the astatine atom and/or radiolytic effects of ²¹¹At α-particles could have reduced binding affinity. Nevertheless, the binding affinities for iso-[²¹¹At]SAGMB-5F7 (3.0±0.1 nM) and [²¹¹At]SAGMB-5F7 (4.5±0.4 nM) should be compatible with their use as targeted radiotherapeutics.

Maximizing radionuclide trapping in cancer cells after binding and cellular processing of radiolabeled receptor-targeted proteins should increase effectiveness for targeted radiotherapy. Internalization assays performed with both trastuzumab and 5F7 VHH demonstrated that labeling these HER2-targeted proteins with either [*I]SGMIB or iso-[*I]SGMIB resulted in a similar degree of cellular trapping of radioiodine up to 6 h; however, at 24 h, total cell associated and internalized activities were significantly lower for the iso-[*I]SGMIB conjugates. Sec Choi J. Vaidyanathan G, Koumarianou E. McDougald D. Pruszynski M. Osada T. et al. N-Succinimidyl guanidinomethyl iodobenzoate protein radiohalogenation agents: influence of isomeric substitution on radiolabeling and target cell residualization. Nucl Med Biol 2014; 41:802-12, which is incorporated herein by reference. Although these results suggest that the residualizing capability of iso-[*I]SGMIB is not as prolonged as that of [*I]SGMIB, this might not be a significant disadvantage with ²¹¹At because of its 7.2-h half-life. Paired label experiments on BT474M1 breast carcinoma cells permitted direct comparison of cell associated and intracellular activity for both iso-[²¹¹At]SAGMB-5F7 and [²¹¹At]SAGMB-5F7 to their radioiodinated counterparts. Our results indicated that astatine-for-iodine substitution had no effect on residualizing capacity with both the 1.3.4- and 1.3.5-isomers; however, for the latter, a significant decrease in intracellular trapping was observed with both iso-[²¹¹At]SAGMB-5F7 and iso-[¹³¹I]SGMIB-5F7 at 24 h. Although the mechanism responsible for this behavior is not known, it seems likely that a higher rate of catabolismand/or egress of labeled catabolites for the 1.3.5-isomers could play a role. Nonetheless, even with iso-[²¹¹At]SAGMB-5F7. 48.4±5.5% of initially bound radioactivity remained internalized at 24 h, which is encouraging because more than 90% of ²¹¹At atoms would have decayed by this time.

The primary focus of this study was the evaluation of the ²¹¹At-labeled 5F7 VHH conjugates which to the best of our knowledge, represents the first attempt to evaluate this promising α-emitter for labeling VHH molecules. One of these agents. [²¹¹At]SAGMB, has been used successfully for labeling the internalizing intact mAb L8A4 that reacts with a mutant form of the epidermal growth factor receptor. However, extrapolation of results from one type of protein construct to another must be done with caution. For example, N^{ε}-(3-[¹³¹I]iodobenzoyl)-Lys⁵-N^{α}-malcimido-Gly¹-GEEEK (¹³¹I-IB-Mal-D-GEEEK) was shown to be an excellent reagent for labeling intact mAb L8A4 but offered no advantages in terms of tumor uptake, and a distinct disadvantage in terms of kidney uptake. for labeling 5F7 VHH. Importantly, the high and prolonged retention of radioactivity in HER2-expressing BT474M1 cancer cells observed in the internalization assays with [²¹¹At]SAGMB-5F7 and iso-[²¹¹At]SAGMB-5F7 was replicated in the paired-label biodistribution studies performed in SCID mice with xenografts derived from the same BT474M1 cell line. The magnitude of tumor accumulation observed with these ²¹¹At-labeled 5F7 conjugates was two- to threefold higher than reported for another HER2-targeted VHH. 2Rs15d, labeled with ^{99m}Tc, ¹⁷⁷Lu, ⁶⁸Ga and ¹⁸F as well as HER2-specific affibodies labeled with a variety of radionuclides including ²¹¹At.

Regarding the possibility of isomer substitution pattern affecting tumor activity levels, iso-[¹³¹I]SGMIB-5F7 and iso-[²¹¹At]SAGMB-5F7 exhibited a significant and unexpected ˜1.5-fold tumor delivery advantage compared with [¹³¹I]SGMIB-5F7 and [²¹¹At]SAGMB-5F7 at all time points. However, this does not appear to reflect differences in residualization capacity because similar degrees of intracellular trapping were observed for both isomers in the in vitro internalization assays until the last time point. With regard to differences in the in vivo behavior of the ²¹¹At- and ¹³¹I-labeled VHH conjugates, the localization of [²¹¹At]SAGMB-5F7 and iso-[²¹¹At]SAGMB-5F7 in HER2-positive BT474M1 xenografts was comparable to that of their co-administered ¹³¹I-labeled analogues at early time points but about 20% lower at 21 h. This likely reflects halogen-dependent differences in in vivo stability. with a higher rate of dehalogenation for astatine the most probable cause, consistent with the lower C-X bond strength for astatine. This is supported by the observation of higher levels of ²¹¹At compared with 1311 in thyroid and stomach, tissues known to sequester free radiohalides, with both isomers. However, activity levels in the thyroid and stomach after injection of [²¹¹At]SAGMB-5F7 were 0.4-0.6% and 1.0-2.3% ID, respectively. while those for iso-[²¹¹At]SAGMB-5F7 were 0.2-0.3% and 0.6-1.7% ID, respectively, suggesting a lower degree of deastatination for the iso-[²¹¹At]SAGMB conjugate. Likewise, stomach and thyroid radioactivity levels after injection of iso-[¹³¹I]SGMIB-5F7 were lower than those for [¹³¹I]SGMIB-5F7, suggesting unexpected isomer-dependent differences in the in vivo stability of these radiohalogenated sdAb conjugates. Nonetheless, the degree of ²¹¹At uptake in thyroid and stomach for both [²¹¹At]SAGMB-5F7 and iso-[²¹¹At]SAGMB-5F7 were lower than those reported for a variety of lower molecular weight proteins labeled using several different methods. Even though the loss of ²¹¹At[astatide] from [²¹¹At]SAGMB-5F7 and iso-[²¹¹At]SAGMB-5F7 was relatively low, it could increase normal tissue toxicity, which can be reduced significantly through the use of blocking agents as was done in clinical studies with ²¹¹At-labeled antibodies.

Tumor-to-normal tissue ratios were generally higher for the radioiodinated conjugates compared with the astatinated versions, presumably reflecting the higher in vivo stability of the iodo versions. Unexpectedly, tumor-to-normal tissue ratios were significantly higher with both radionuclides when 5F7 VHH was labeled using the iso-prosthetic agents. As summarized in Tables 1 and 2, this reflects not only some advantages in tumor uptake but also considerably lower activity levels in normal tissues. particularly with the ¹³¹I-labeled conjugates. A possible explanation for this behavior is a mass effect wherein a certain mass of VHH molecule is needed to block nonspecific uptake of the labeled VHH in normal organs such as the liver spleen and lungs. See Xavier C. Vaneycken I. D'Huyvetter M. Heemskerk J. Keyaerts M. Vincke C. et al. Synthesis. preclinical validation, dosimetry, and toxicity of ⁶⁸Ga-NOTA-anti-HER2 nanobodies for iPET imaging of HER2 receptor expression in cancer. J Nucl Med 2013; 54:776-784, which is incorporated herein by reference. This could be relevant here because the [²¹¹At]SAGMB-5F7 plus [¹³¹I]SGMIB-5F7 biodistribution experiment was performed at a total 5F7 VHH dose of 2.1 μg while in the iso-[²¹¹At]SAGMB-5F7 plus iso-[¹³¹I]SGMIB-5F7 study, a total 5F7 dose of 4.3 μg was administered. However, this is likely not a factor because the biodistribution observed for [¹³¹I]SGMIB-5F7 in the current study at a total VHH dose of 2.1 μg was quite similar to those reported previously for [¹³¹I]SGMIB-5F7 at total 5F7 doses of 4.3. and 6.8 μg. See Vaidyanathan G. McDougald D. Choi J., Koumarianou E. Weitzel D. Osada T. et al. Preclinical evaluation of ¹⁸F-labeled anti-HER2 nanobody conjugates for imaging HER2 receptor expression by immuno-PET. J Nucl Med 2016; 57:967-73, which is incorporated herein by reference. Moreover, significant mass dependent localization differences were observed for the anti-HER2 VHH 2Rs15d after labeling with ⁶⁸Ga between 0.1 and 1 μg doses but not between doses of 1 and 10 μg. which encompasses the doses used in the current study.

The differences observed in the biological behavior with the two isomer versions with the same radiohalogen were unexpected, particularly given the similarity in tissue distribution observed previously when iso-[¹²⁵I]SGMIB-trastuzumab and [¹³¹I]SGMIB-trastuzumab were compared in the same animal model. See Choi J. et al., Nucl Med Biol 2014; 41:802-12, which is incorporated herein by reference. However, VHH molecules are about 10 times smaller than intact mAbs, which may lead to more rapid degradation to species that are small enough to allow easy access to deiodinases and other enzymes such as cytochrome P450 that can lead to dehalogenation. The greater metabolic stability of iso-[¹²⁵I]SGMIB-5F7 compared with [¹³¹I]SGMIB-5F7 could be explained by differences in the catabolismof the two conjugates and the susceptibility of the labeled catabolites towards in vivo deiodination. As summarized in a recent review, subtle differences in the design of radioiodinated compounds can lead to increased rates of deiodination. Consistent with this, the deiodination of meta-iodobenzylguanidine (structural element of iso-SGMIB) was less than that of ortho-iodobenzylguanidine (structural element of SGMIB). Studies are planned to evaluate the chemical nature of the labeled catabolites generated from iso-SGMIB-VHH and SGMIB-VHH conjugates to better understand the mechanisms that result in the differences observed in their in vivo behavior.

A potential problem with using VHH molecules as a platform for targeted radiotherapeutics is the high accumulation and prolonged retention of radioactivity in the kidney, which could result in dose limiting renal toxicity. This behavior has been observed with radiometals such as ¹⁷⁷Lu as well as with some residualizing radiohalogenation agents such as ¹³¹I-IB-Mal-D-GEEEK. For example, when 5F7-GGC was labeled using ¹³¹I-IB-Mal-D-GEEEK, kidney levels were greater than 150% ID/g from 1-8 h after injection and about 100% ID/g at 24 h. In contrast, with all four radiohalogenated 5F7 conjugates evaluated in the current study, initial kidney radioactivity levels were high (60-100% ID/g) but decreased rapidly with renal clearance half-lives of about 1-2 h. Surprisingly, renal radioactivity levels for both the ¹³¹I- and ²¹¹At-labeled iso-conjugates were significantly lower than those observed for their corresponding 1,3,4-isomer conjugates at all time points with the difference in kidney retention increasing with time. For example, the renal radioactivity level observed 21 h after injection of iso-[¹³¹I]SGMIB-5F7 was more than 4 times lower than that for [¹³¹I]SGMIB-5F7. Radionuclide-dependent differences in kidney activity levels also were observed although to a lesser extent than those between the two isomeric versions for a given radionuclide. Paradoxically, kidney radioactivity levels after injection of iso-[²¹¹At]SAGMB-5F7 were higher than those for co-administered iso-[¹³¹I]SGMIB-5F7 while renal radioactivity levels after injection of [²¹¹At]SAGMB-5F7 were lower than those for co-administered [¹³¹I]SGMIB-5F7. The differences in renal uptake and retention of the four 5F7 VHH radioconjugates cannot be explained at this time and were unexpected considering the similarity of the acylation agents in physical properties that might influence kidney retention such as polarity and hydrophilicity. Moreover, previous studies showed no significant differences between kidney uptake values for intact mAb L8A4 labeled with [¹³¹I]SGMIB and [²¹¹At]SAGMB, and trastuzumab labeled using [¹³¹I]SGMIB and iso-[¹²⁵I] SGMIB. Although the mechanism(s) responsible for their lower kidney radioactivity levels are not clear, the iso-[²¹¹At]SAGMB and iso-[¹³¹I]SGMIB conjugates are the reagents of choice for minimizing radiation absorbed dose to the kidneys with 5F7 and potentially other VHH. If further reduction in renal radiation dose is needed, it has been shown that this can be accomplished, at least with a ¹⁷⁷Lu-labeled VHH conjugate by co-infusion with the plasma expander Gelofusin.

In summary, it was demonstrated that the anti-HER2 5F7 VHH can be labeled with ²¹¹At in reasonable yields with excellent retention of affinity and immunoreactivity after labeling. Studies in preclinical models with [²¹¹At]SAGMB-5F7 demonstrated high and prolonged tumor targeting and rapid normal tissue clearance, with even more favorable observed with iso-[²¹¹At]SAGMB-5F7. Moreover, iso-[¹³¹I]SGMIB-5F7 was shown to offer significantly improved tumor targeting compared with [¹³¹I]SGMIB-5F7. Taken together, our results suggest that iso-[²¹¹At]SAGMB-5F7 and iso-[¹³¹I]SGMIB-5F7 warrant further evaluation as α-particle and β-particle emitting targeted radiotherapeutics for the treatment of HER2 expressing malignancies.

VHH sequences that target HER2 that are useful in the practice of the invention include those set forth in SEQ ID NOs: 1-5.

immunoglobin heavy chain variable region,
partial [Camelus dromedarius]
SEQ ID NO: 1
DVQLVESGGGSVQGAAGGSLRLSCAASDITYSTDCMGWFRQAPGKER
EGVATINNGRAITYYADSVKGRFTISQDNAKNTVYLQMNSLRPKDTA
IYYCAARLRAGYCYPADYSMDYWGKGTQVTVSS
immunoglobin heavy chain variable region,
partial [Camelus dromedarius]
SEQ ID NO: 2
DVQLEESGGGSVQAGGSLRLSCAASGYIYSTYCMGWFRQAPGKEREG
VAAINDVGGSVYYADSVKGRFTISQDIAQDTMYLQMNDLTPENTVTY
TCAALRCLSDSDPDTRVHMYYDWGQGTQVTVSS
immunoglobin heavy chain variable region,
partial [Camelus dromedarius]
SEQ ID NO: 3
DVQLEESGGGSVQTGGSLRLSCAASGYTYSSACMGWFRQGPGKEREA
VADVNTGGRRTYYADSVKGRFTISQDNTKDMRYLQMNNLKPEDTATY
YCATGPRRRDYGLGPCDYNYWGQGTQVTVSS
immunoglobin heavy chain variable region,
partial [Camelus dromedarius]
SEQ ID NO: 4
EVQLEESGGGLVQPGGSLTLSCAASGYTFTNCAAGWYRQAPGKECEL
VASIFSGNRTNYADSVKGRFTISRDNTKDIVYLQMNSLKPEDTTVYY
CDARTPCWGQGTQVTVSS
immunoglobin heavy chain variable region,
partial [Camelus dromedarius]
SEQ ID NO: 5
EVQLEESGGGSVQAGGSLRLSCAASGYTFLQLLHGWFRQAPGKEREV
VARFNTDINKTFYLESVKGRFTLSQDNAKNTLYLQMNSLKPEDTAIY
YCAASRPDSTCDYFAYRGQGTQVTVSS

All publications, patents and patent applications mentioned in the specification are indicative of the level of those skilled in the art to which this invention pertains. All publications, patents and patent applications are herein incorporated by reference to the same extent as if each individual publication, patent or patent application was specifically and individually indicated to be incorporated by reference. Although the foregoing invention has been described in some detail by way of illustration and example for purposes of clarity of understanding, it will be obvious that certain changes and modifications may be practiced within the scope of the embodiments.

What is claimed is:

1. A compound in the form of a prosthetic compound or radiohalogen precursor represented by Formula I: 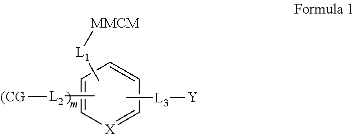
wherein:
X is CH or N;
L₁ and L₃ are independently selected from a bond, a substituted or unsubstituted alkyl chain, a substituted or unsubstituted alkenyl chain, a substituted or unsubstituted alkynyl chain, and a polyethylene glycol (PEG) chain;
MMCM is a macromolecule conjugating moiety;
L₂ is a substituted or unsubstituted alkyl chain, a substituted or unsubstituted alkenyl chain, a substituted or unsubstituted alkynyl chain, or a polyethylene glycol (PEG) chain comprising at least three oxygen atoms, wherein L₂ optionally contains a Brush Border enzyme-cleavable peptide;
CG is selected from guanidine; PO₃H; SO₃H; one or more charged D- or L-amino acids selected from arginine, phosphono/sulfo phenylalanine, glutamate, aspartate, and lysine; a hydrophilic carbohydrate moiety; a polyethylene glycol (PEG) chain; and Z-guanidine;
Z is (CH₂)ₙ;
n is greater than 1;
m is 0 to 3; and
Y is an alkyl metal moiety or a radioactive halogen selected from the group consisting of ⁷⁵Br, ⁷⁶Br, ⁷⁷Br, ¹²³I, ¹²⁴I, ¹²⁵I, ¹³¹I, and ²¹¹At, or a pharmaceutically acceptable salt or solvate thereof.

2. The compound of claim 1, wherein MMCM is selected from the group consisting of N-hydroxysuccinimide (NHS) ester, tetrafluorophenol (TFP) ester, an isothiocyanate group, or a maleimide group.

3. The compound of claim 1, wherein L₂ is (CH₂)ₚ, wherein p=1 to 6.

4. The compound of claim 1, wherein the optional Brush Border enzyme-cleavable peptide is selected from the group consisting of Gly-Lys, Gly-Tyr and Gly-Phe-Lys.

5. The compound of claim 1, represented by the following structure:

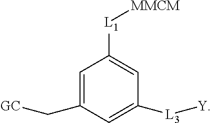

6. The compound of claim 5, wherein the compound comprises N-succinimidyl 3-guanidinomethyl-5-[¹³¹I]iodobenzoate, or N-succinimidyl 3-[²¹¹At]astato-5-guanidinomethyl benzoate.

7. A radiolabeled biomolecule or intermediate, comprising the compound of claim 1 attached to a biomolecule.

8. The radiolabeled biomolecule or intermediate of claim 7, wherein the biomolecule is selected from the group consisting of an antibody, an antibody fragment, a VHH molecule, an aptamer or variations thereof.

9. The radiolabeled biomolecule or intermediate of claim 7, wherein said labeled biomolecule is a VHH.

10. The radiolabeled biomolecule or intermediate of claim 9, wherein said VHH targets HER2.

11. A pharmaceutical composition comprising the radiolabeled biomolecule of claim 7, in association with a pharmaceutically acceptable adjuvant, diluent or carrier.

12. A compound in the form of a prosthetic compound or radiohalogen precursor represented by Formula 2:
MC—Cm—L₄—Cm—T Formula 2,
wherein:
MC is a polydentate metal chelating moiety;
Cm is thiourea, amide, or thioether;
L₄ is selected from a bond, a substituted or unsubstituted alkyl chain, a substituted or unsubstituted alkenyl chain, a substituted or unsubstituted alkynyl chain optionally having NH, CO, or S on one or both termini, and a polyethylene glycol (PEG) chain;
T is the compound of claim 1,
or a pharmaceutically acceptable salt or solvate thereof.

13. A radiolabeled biomolecule or intermediate, comprising the compound of claim 12, attached to a biomolecule.

14. The radiolabeled biomolecule or intermediate of claim 13, wherein the biomolecule is selected from the group consisting of an antibody, an antibody fragment, a VHH molecule and an aptamer.

15. The radiolabeled biomolecule or intermediate of claim 13, wherein said biomolecule is a VHH.

16. The radiolabeled biomolecule or intermediate of claim 15, wherein said VHH targets HER2.

17. A pharmaceutical composition comprising the radiolabeled biomolecule of claim 13, in association with a pharmaceutically acceptable adjuvant, diluent, or carrier.

18. A method of treatment for cancer comprising administering to an individual in need thereof an effective amount of the radiolabeled biomolecule of claim 7.

19. A method of treatment for cancer comprising administering to an individual in need thereof an effective amount of the radiolabeled biomolecule of claim 13.
